(19) United States
(12) Patent Application Publication
(10) Pub. No.: US 2009/0104228 A1
(43) Pub. Date: Apr. 23, 2009

(54) Influenza Virus Vaccine

(75) Inventors: Larisa Georgievna Rudenko, St Petersburg (RU); Julia Desheva, St. Petersburg (RU); Galina Ibragimovna Alexandrova, St. Petersburg (RU)

Correspondence Address: STERNE, KESSLER, GOLDSTEIN & FOX P.L.L.C., 1100 NEW YORK AVENUE, N.W., WASHINGTON, DC, 20005, US

(73) Assignee: BioDiem Ltd, Melbourne Victoria (AU)

(21) Appl. No.: 12/297,537

(22) PCT Filed: Apr. 18, 2007
(86) PCT No.: PCT/AU2007/000501

§ 371 Date: Oct. 17, 2008

(30) Foreign Application Priority Data
Apr. 19, 2006 (RU) 2006113251

(51) Int. Cl. A61K 39/145; C12N 7/01; C12N 7/04

(52) U.S. Cl. 424/206.1; 435/235.1; 435/236

(57) ABSTRACT

The invention relates to influenza virus vaccines, and in particular to a reassortant influenza virus which has at least its hemagglutinin gene derived from a non-pathogenic or low pathogenic influenza virus, and its other genes derived from a donor strain. In one embodiment the influenza virus is a 7:1 reassortant, in which only the hemagglutinin gene is derived from a non-pathogenic influenza virus. The virus is useful for production of vaccines against influenza, including influenza caused by highly pathogenic influenza virus strains.

### PRIORITY

This application claims priority from Russian patent application No. 2006113251 dated 19 Apr. 2006, the entire disclosure of which is incorporated herein by this reference.

### FIELD

This invention relates to vaccines against influenza virus, and in particular to vaccines against highly-pathogenic avian influenza virus. In one embodiment the invention provides influenza virus strains useful in the production of a live attenuated intranasal vaccine or a parenteral inactivated influenza vaccine.

### BACKGROUND

All references, including any patents or patent application, cited in this specification are hereby incorporated by reference to enable full understanding of the invention. Nevertheless, such references are not to be read as constituting an admission that any of these documents forms part of the common general knowledge in the art, in Australia or in any other country. The discussion of the references states what their authors assert, and the applicants reserve the right to challenge the accuracy and pertinency of the cited documents.

Repeated outbreaks of highly pathogenic avian influenza (HPAI) H5NI virus in domestic poultry and wild birds in Asia continue to pose a pandemic threat to human health. HPAI viruses of serotype H5N1 were first recognized to cause respiratory disease in humans in Hong Kong in 1997, when viruses from infected poultry caused 18 documented human cases, including six fatalities. In 2003, H5N1 virus reemerged in humans to infect two family members in Hong Kong, resulting in the death of one person. Since late 2003, unprecedented numbers of HPAI H5N1 outbreaks in poultry have occurred in many Asian, European and African countries, resulting in more than 220 laboratory-confirmed human cases in Hong Kong, Vietnam, Thailand, Cambodia, and Indonesia, with a fatality rate of greater than 50%. So far infection has been transmitted from birds or animals to humans; there has not been any confirmed instance of significant human-human transmission, other than perhaps between immediate relatives. However, if the virus develops the ability to pass from human to human a pandemic could rapidly develop.

Despite rigorous attempts by health authorities, local groups and farmers in many countries to contain outbreaks of avian influenza among poultry by killing infected birds and inoculating healthy ones, there are some countries where there is the potential for outbreaks to spread and to be transmitted to humans. Thus there is still the danger of an epidemic or pandemic.

The influenza virus sub-types H1N1 and H3N2 which are currently used for vaccination against epidemic or seasonal influenza A cannot generate a strong protective reaction in case of a large scale outbreak caused by viruses of sub-type H5N1, to which the majority of the population is not immune.

Currently available intramuscular inactivated influenza vaccines (IIV) are effective in inducing relatively strain-specific neutralizing serum antibodies, but are less effective in inducing secretory IgA in nasal wash fluids. In contrast, intranasally (i.n.) delivered live attenuated influenza vaccines (LAIV) elicit systemic and mucosal immune responses as well as cell-mediated immunity. Since mucosal IgA responses have been shown to exhibit heterotypic cross-reactivity, LAIV may offer broader protection against heterologous strains.

Since 1997, HPAI H5N1 viruses from birds have undergone rapid genetic evolution. The viruses isolated from humans have reflected this genetic variation, with concomitant antigenic variation. H5N1 viruses from 2004 to 2005 comprise two genetically distinct virus clades, both of which are antigenically distinct from the 2003 human isolates, which in turn were antigenically distinct from those isolated from humans in 1997. Once recognized to cause human disease, new candidate vaccine strains must be generated for each H5N1 antigenic variant. Because of this antigenic heterogeneity, vaccines which provide broader cross-protective immunity against antigenically distinct H5N1 viruses are highly desirable.

A number of different strategies have been applied to generate vaccine candidates against HPAI H5N1 viruses, including the use of antigenically-related non-pathogenic viruses to produce an IIV, and the use of purified recombinant hemagglutinin (HA) protein. Both of these approaches have been evaluated clinically, with suboptimal results. More recently, reverse genetics techniques have been optimized to allow for the generation of vaccine reassortant strains which possess HA with the modified multibasic cleavage site which is associated with virulence in birds, and internal genes derived from a human vaccine donor strain. This approach allows for the inclusion of an HA protein, albeit modified, which is antigenically closely related to that found in the circulating HPAI H5N1 virus.

Development of an LAIV for pandemic preparedness has certain advantages over other vaccine strategies. Since LAIV may provide effective protection against a broader range of variants, an exact match between the vaccine strain and circulating viruses may be less critical. As an example, LAIV was shown to provide highly effective protection in healthy pre-school children against a drift variant of influenza A (H3N2) in a clinical trial of LAIV in the United States. Similar data have been obtained in Russia. The heterotypic efficacy of LAIV may be at least in part due to the induction of enhanced IgA antibody responses in the respiratory tract compared with those induced by IIV. Furthermore, since vaccine will be in short supply during a pandemic, multiple vaccine production options may be important.

Although a number of different vaccines against avian influenza are in pre-clinical development or in clinical trial in humans, so far only one has been approved for use in the United States. However, this vaccine, produced by Sanofi-Aventis SA, elicits a protective immune response in only 54% of adults who receive the vaccination, compared to the 75-90% protection against normal seasonal influenza strains conferred by seasonal vaccines. The results showed that of the subjects who received two injections of the highest dose, 90 μg, only 45% developed levels of antibodies sufficient to be considered protective against the virus.

Most of the vaccines against avian influenza which are currently in development are prepared from HPAI H51N strains, and therefore require the use of high-level containment facilities and rigorous precautions to ensure that the vaccine does not contain viable pathogenic virus. This contributes substantially to the difficulty and cost of vaccine development.

There is therefore a need for alternative vaccines which may provide a greater level of protection against avian influenza. In particular the development of safe, dose-sparing and effective human vaccines against H5N1 influenza is a high priority for global public health.

### SUMMARY

In a first aspect, the invention provides a reassortant influenza virus which comprises a hemagglutinin gene derived from a non-pathogenic or low pathogenic influenza virus, and its other genes derived from a donor strain, in which the non-pathogenic or low pathogenic influenza virus has the same hemagglutinin type as that of the highly pathogenic influenza virus.

In some embodiments the non-pathogenic or low pathogenic influenza virus is an avian virus.

In some embodiments the virus is a 7:1 reassortant, in which only the hemagglutinin gene is derived from a non-pathogenic influenza virus.

In a second aspect, the invention provides a vaccine against a highly pathogenic influenza virus, comprising

a) a reassortant influenza virus comprising a hemagglutinin gene derived from a non-pathogenic avian influenza virus, and
b) other genes derived from a donor strain.

In a third aspect, the invention provides a method for preparing a vaccine for immunization of a subject against an avian influenza virus strain, comprising the step of mixing an influenza virus according to the first aspect of the invention with a carrier, and optionally with one or more additional influenza viruses and/or an adjuvant.

In a fourth aspect the invention provides a method for protecting a subject against infection with a highly pathogenic influenza virus, comprising the step of immunizing the subject with a vaccine according to the second aspect of the invention.

In a fifth aspect the invention provides the use of an influenza virus according to the first aspect of the invention in the manufacture of a vaccine for immunization of a subject against a highly pathogenic influenza virus strain.

In the fourth and fifth aspects the vaccine may be an LAIV or an IIV. When the vaccine is a LAIV, it may be formulated for oral or intranasal administration.

In one embodiment of the fourth and fifth aspects of the invention the vaccine provides cross-protection and/or a cross-reactive immune response against a highly pathogenic influenza virus strain.

We have prepared a candidate H5 pandemic 7:1 reassortant vaccine from an antigenically related non-pathogenic avian influenza H5N2 and a cold-adapted (ca) influenza donor strain A/Leningrad134/17/57 (H2N2; Len17) using classical reassortment techniques. This candidate vaccine has been evaluated for its protective efficacy against antigenically heterologous HPAI H5N1 strains. The H5 pandemic vaccine candidate (Len 17/H5) derives its HA from non-pathogenic A/Duck/Potsdam/1402-6/86 (H5N2; Pot/86) virus, and all its other genes from Len17 (7:1 reassortant). Pot/86 virus is antigenically similar to the 1997 H5N1 viruses isolated from humans. We compared H5 cross-reactive immunity and protective efficacy against a contemporary H5N1 strain A/Vietnam/1203/2004 (VN/1203) induced by LAV and IIV prepared from this reassortant virus, or by an IIV generated against another H5N1 strain, A/Hong Kong/213/2003 (HK213), which was the HA and NA donor for the 2003 H5N1 vaccine candidate.

Len17/H5 demonstrated ca and ts phenotypes in vitro similar to those of the Len17 ca donor strain, grew to high titres in embryonated eggs, and shared antigenic similarity with the H5N1 viruses isolated from humans in 1997. We demonstrate herein that the reassortant Len17/H5 virus is attenuated in mice and non-infectious for chickens, and effectively protects mice against heterologous HPAI H5N1 infection when used as either an LAIV or IIV. The Len17/H5 vaccine candidate also possessed the high-growth properties in embryonated eggs which are desirable for the production of IIV.

As an LAIV, a single dose of Len17/H5 induced superior H5 virus-specific IgA antibody responses in the respiratory tract, whereas a single dose of Len17/H5 IIV induced better cross-reactive serum neutralizing and IgG antibody responses to HK/156 virus HA. Surprisingly, a single dose of Len17/H5 administered either as an LAIV or IIV elicited protective immunity in mice against both related and antigenically variant H5N1 viruses.

These results suggest a pandemic vaccine strategy which does not require reverse genetics technology, rigorous bio-safety precautions, or a precise antigenic match for vaccine strain generation, yet may offer protection against a heterologous virus in the early phase of a pandemic. The use of a non-pathogenic H5 virus to generate the Len17/115 vaccine strain by traditional reassortment methods may be an advantage in countries which have limited containment laboratory capacity or access to the patented reverse genetics technology required to derive vaccine strains from HPAI H5 viruses. The reassortant viruses according to the invention can be produced by classical methods, and therefore avoid the need to resort to reverse genetics strategies. Furthermore, the lack of virus replication or induction of virus-specific antibody in chickens inoculated with Len17/H5 suggests that the large-scale manufacturing of a non-pathogenic H5 reassortant vaccine strain would not pose any threat to the poultry industry.

### BRIEF DESCRIPTION OF THE FIGURES

FIG. 1 illustrates the anti-HK/156 HA-specific antibody responses in mice immunized with H5 vaccine. Mice were infected i.n. with one dose of 300 MID₅₀ of Len17/H5 LAIV or injected i.m. with one dose of 10 μg of Len17/H5 IIV. Two groups of mice were infected i.n. with either 300 MID₅₀ of Pot/86 wild-type or 100 MID₅₀ of HK/213 virus as positive controls. Mice received PBS as a negative control. Serum (A), lung (B), and nasal washes (C) were collected 6 weeks after vaccination or infection, and were tested by ELISA for the presence of IgG and IgA antibody, using a purified HK/156 recombinant HA protein as antigen. Values are the mean (log₁₀) c S.D. of reciprocal end-point titres of five mice per group. * p<0.05 compared with Len17/H5 IIV group or † p<0.05 compared with Len17/H5 LAIV group.

FIG. 2 shows anti-VN/1203 HA-specific antibody responses in H5 vaccinated mice. Five mice per group were immunized once with Len17/H5 or Len17 (H2N2) LAIV or IIV, or were inoculated i.n. with live HK/213 virus or PBS. Serum (A) and nasal washes (B) were collected 6 weeks after vaccination or infection, and tested by ELISA for the presence of (A) IgG1, IgG2a, or (B) IgG and IgA antibody, using a purified VN/1203 rHA protein as antigen. Values are the mean (log₁₀)±S.D. of reciprocal end-point titres of five mice per group. * p<0.05 compared with PBS group.

FIG. 3 shows the induction of influenza H5N1 virus-specific cytokines by LAIV or IIV. Five mice per group were immunized once with Len17/H5 or Len17 (H2N2) LAIV or IIV, or were inoculated i.n. with live HK/213 virus or PBS. Six weeks later, single cell suspensions of spleen were stimulated with either five HAU of inactivated H5N1 whole virus or 250 ng of H5 rHA. Culture supernatants were harvested after 5 days, and cytokines were detected using the Bio-Plex assay.

### DETAILED DESCRIPTION

The highly pathogenic influenza virus against which the vaccine provides cross-protection may be of any hemagglutinin type, including H1, H2, H3, H4, H5, H6, H7, H8, H9, H10, H11, H12, H13, H14, H15 or H16.

The highly pathogenic influenza virus may be one of any sub-type, including but not limited to H5N1, H5N2, H5N8, H5N9, H7N3, H7N7, and H9N2.

Any non-pathogenic or low pathogenic influenza virus may be used, provided that it has the same hemagglutinin type as that of the highly pathogenic influenza virus. In some embodiments the non-pathogenic or low pathogenic influenza virus is an avian virus. In one embodiment the non-pathogenic or low pathogenic avian influenza virus is A/Duck/Potsdam/1042-6/86 (H5N2) A/Vietnam/1194/04(H5N1), A/Duck/Singapore/97 (H5N3), A/Duck/Hokkaido/67/96 (H5N4) or A/Mallard/Netherlands/12/00 (H7N3).

The non-pathogenic or low pathogenic avian influenza virus may be isolated from any wild or domesticated bird, including but not limited to chickens, turkeys, ducks, geese, swans, and other waterbirds.

The donor strain should be of a hemagglutinin type which is different from that of the non-pathogenic influenza virus, because if it is of the same hemagglutinin type it is very difficult to identify reassortants. In some embodiments the donor strain is one of type H2N2 or H1N1.

It is advantageous from the safety and regulatory point of view to use a donor strain which is a fully characterized vaccine strain, and in some embodiments this may be a cold-adapted or temperature-sensitive strain. In some embodiments the donor strain is cold-adapted and temperature-sensitive.

Suitable donor strains include

A/Leningrad/134/17/57 (H2N2)

A/Leningrad/134/47/57 (H2N2)

A/Leningrad/134/17/K7/57 (H2N2)

A/Moscow/21/65 (H2N2)

A/Moscow/21/17/65 (H2N2)

A/Ann Arbor/6/60 (H2N2)

A/Puerto Rico/8/34 (H1N1)

A/Puerto Rico/8/59/1 (H1N1)

In some embodiments, the first aspect of the invention is directed to a new antigenic variant of an influenza virus vaccine strain which uses A/Leningrad/134/17/57(H2N2) as a cold-adapted attenuation donor and a non-pathogenic A/Duck/Potsdam/1402-6/86(H5N2) virus of avian influenza as a source of surface antigens. The attenuation donor A/Leningrad/134/17/57(H2N2) is a cold-adapted temperature-sensitive strain of influenza virus approved in Russia for production of intranasal influenza vaccines for adults and children (Alexandrova, 1986).

For example the vaccine strains A/17/New Calcdonia/99/145(H1N1) (Russian Patent No. 2183672, published on 20 Jun. 2002) and A/17/Panama/99/242(H3N2) (Russian Patent No. 2248935, published on 20 Mar. 2005) are also suitable for use as attenuation donors.

We have prepared a cold-adapted master donor strain which is designated influenza virus A/PR/8/59/1. This has mutations in the PB2, PA, NA and M genes, like those in our other master donor strain influenza virus A/Len/134/47/57(H2N2), and we have used this strain to develop reassortants such as influenza virus A/F/2/82(H3N2) and A/Len/234/84(H1N1) for use in LAIV and inactivated vaccines (Alexandrova, 1989).

The Leningrad and Moscow strains referred to above are cold-adapted and temperature-sensitive, and have been extensively used in Russia for production of LAIVs. The PR and Ann Arbor strains have been used for production of IIVs and LAIVs respectively in the United States.

The viruses for reassortment may be grown in any suitable host. Growth in embryonated chicken eggs is very widely used. Alternatively the viruses may be grown in cell cultures. A wide variety of host cells is suitable, including mammalian cell lines such as Madin-Darby canine kidney cells (MDCK cells) Vero cells (African green monkey kidney cells), BHK (baby hamster kidney) cells, primary chick kidney (PCK) cells, Madin-Darby Bovine Kidney (MDBK) cells, 293 cells (e.g. 293T cells), and COS cells (e.g. COS1 or COS7 cells). See for example WO 97/37000, WO 97/37001, and WO 2005/10779. PBS-1 cells (HepaLife Technologies, Inc) have been reported to provide superior yields of avian influenza virus; see U.S. Pat. No. 5,989,805 (“Immortal Avian Cell Line To Grow Avian and Animal Viruses To Produce Vaccines”), U.S. Pat. No. 5,827,738, U.S. Pat. No. 5,833,980, U.S. Pat. No. 5,866,117 and U.S. Pat. No. 5,874,303. Avian cell lines such as EBx™ cells (Vivalis, Nantes, France) or chicken fibroblasts may also be used.

The reassortant may be prepared by conventional methods, such as that of Ghendon et al (1984), or may be prepared by the reverse genetics method disclosed in WO 91/03552 and U.S. Pat. No. 5,166,057. Plasmid-based reverse genetics techniques are disclosed in WO 00/60050, WO 01/04333 and U.S. Pat. No. 6,649,372, and anti-sense methods are disclosed in WO 00/53786.

The vaccine may be of any kind, including but not limited to live attenuated vaccine (LAIV), inactivated vaccine (IIV; killed virus vaccine), subunit (split vaccine); sub-virion vaccine); purified protein vaccine; or DNA vaccine. Methods for production of all of these types of vaccines are very well known in the art.

In some embodiments the vaccine is a live attenuated vaccine (LAIV), and may be in a formulation suitable for intranasal administration.

The vaccine may also comprise

(a) one or more additional influenza viruses, and/or
(b) a substantially pure influenza neuraminidase protein and/or influenza hemagglutinin protein.
The other influenza viruses may be current seasonal strains, of the kind used in conventional influenza vaccines. For example two type A strains and one type B strains may be used in addition to the virus of the invention. In one embodiment the neuraminidase and hemagglutinin proteins are the mature glycosylated proteins, and may be either isolated from influenza virions or produced by recombinant methods, for example as described in U.S. Pat. No. 6,485,729.

The vaccine optionally also comprises an adjuvant. Suitable adjuvants which have been used in previous influenza vaccines for humans include alum, oil emulsion compositions such as MF59 (5% squalene, 0.5% Tween 80, 0.5% Span 85; see WO90/14387), saponins such as ISCOMs, or a block copolymer such as CRL 1005 (Katz et al, 2000), and double-stranded RNA, such as Ampligen® Hemisperx Biopharma, Inc). Adjuvants for use with influenza vaccines are also discussed in WO2005/10797 and WO2006/04189.

In some embodiments the vaccine elicits an IgG response, an IgA response and/or a T cell response. In other embodiments the vaccine elicits IgA, IgG and T cell responses.

Suitable carriers are well known in the art. In some embodiments of an LAIV according to the invention the carrier is one which enables the vaccine to be stored at refrigerator temperature so that lyophilization of the vaccine is not required. Such formulations are known for a variety of viruses, including influenza, and typically contain a sugar, an amino acid and a buffer, and may also include a protein such as gelatin or casein, or a derivative thereof. See for example U.S. Pat. No. 4,338,335; Yannarell et al, 2002; Ikizler and Wright, 2002; WO2006/04819; and WO 2005/014862.

In some embodiments the highly pathogenic influenza virus against which the vaccine provides cross-protection is of hemagglutinin type H1, H2, H3, H4, H5, H6, H7, H8, H9, H10, H11, H12, H13, H14, H15 or H16. In some embodiments the highly pathogenic influenza virus against which the vaccine provides cross-protection is of type H5N1, H5N2, H5N8, H5N9, H7N3, H7N7 or H9N2.

While it is particularly contemplated that the vaccines of the invention are suitable for use in medical treatment of humans, they are also applicable to veterinary treatment, including treatment of non-human primates or monkeys.

Methods and pharmaceutical carriers for preparation of vaccines are well known in the art, as set out in textbooks such as Remington's Pharmaceutical Sciences, 20th Edition, Williams & Wilkins, Pennsylvania, USA.

The compounds and compositions of the invention may be administered by any suitable route, and the person skilled in the art will readily be able to determine the most suitable route and dose for the condition to be treated. The dosages to be used for immunization will depend inter alia on the individual vaccine, the route of immunization and the age of the recipient, and can readily be determined in the course of routine clinical trial. Dosages used with seasonal influenza vaccines may be used as a guide.

The carrier or diluent, and other excipients, will depend on the route of administration, and again the person skilled in the art will readily be able to determine the most suitable formulation for each particular case.

The most common influenza vaccines currently used are inactivated vaccines, which may be comprise whole virus particles (virions), virions which have subjected to treatment with agents which dissolve lipids (“split” vaccines), or purified viral glycoproteins (“sub-unit vaccines”). These inactivated vaccines mainly protect by eliciting production of antibodies directed against the hemagglutinin. Antigenic evolution of the influenza virus by mutation results in modifications in HA and NA. Consequently these inactivated vaccines only protect against strains which have surface glycoproteins which comprise identical or cross-reactive epitopes.

To provide a sufficient antigenic spectrum, conventional vaccines comprise components from several viral strains; they generally contain two type A strains and one type B strain. The choice of strains for use in vaccines is reviewed annually for each particular year and is predicated on recommendations provided by the World Health Organization and the United States food and Drug Administration (FDA). These recommendations reflect international epidemiological observations. Viral strains may be obtained from sources such as the National Institute for Biological Standards and Control, London, UK, the World Influenza Centre, London, UK, the Centers for Disease Control, Atlanta, USA, and the Center for Biologics Evaluation and Research, Washington, USA.

The influenza virions consist of an internal ribonucleoprotein core (a helical nucleocapsid) containing the single-stranded RNA genome, and an outer lipoprotein envelope lined inside by a matrix protein (M). The segmented genome of influenza A consists of eight molecules of linear, negative polarity, single-stranded RNAs which encode ten polypeptides, including the RNA-directed RNA polymerase proteins (PB2, PB1 and PA) and nucleoprotein (NP) which form the nucleocapsid; the matrix proteins (M1, M2); two surface glycoproteins, hemagglutinin (HA) and neuraminidase (NA), which project from the lipoprotein envelope; and non-structural proteins whose function is unknown (NS1 and NS2). Transcription and replication of the genome takes place in the nucleus, and assembly occurs via budding on the plasma membrane. The hemagglutinin envelope glycoprotein is involved in cell attachment and entry during infection. The neuraminidase envelope glycoprotein is required for the release of daughter virus particles from the host cell. The influenza viruses can reassort genes when viruses of two or more different strains infect a single host cell or organism.

The two major surface glycoproteins, HA and NA, are highly immunogenic, and are subject to continuous and sequential evolution within immune or partially immune populations. When NA is present in immunogenic form in the vaccine or on the intact virion, it is a minority component, and therefore subservient to continuing antigenic competition with the immunodominant HA. The antibody induced by the HA directly neutralizes virus infectivity; antibody to the NA, while not neutralizing, limits viral replication in a multi-cycle infection and can reduce viral replication below a pathogenic threshold. However, NA can synergistically enhance HA, when the NA is presented in sufficient quantity. It has been reported in U.S. Pat. No. 6,485,729 that the antigenic competition between HA and NA can be wholly or substantially eliminated by presenting the HA and NA as separate purified proteins in a vaccine comprising conventional inactivated influenza virus.

The vaccine strains currently used for preparation of live influenza vaccines (LIV) are obtained by the method of reassortment of contemporary epidemic influenza viruses with cold-adapted (ca) influenza virus donor strains in order to generate reassortants with a mixed genome. The genes encoding hemagglutinin (HA) and neuraminidase (NA) are inherited from the epidemic strain, while the six genes encoding internal and non-structural proteins (PB2, PB1, PA, NP, M, NS) are derived from a harmless HA attenuation donor. Thus these conventional vaccine strains are 6:2 reassortants.

### DEFINITIONS

Influenza is an acute, highly infectious disease caused by the influenza virus. Infection occurs via the respiratory tract, and with seasonal strains recovery is usually quite rapid. However, particularly in elderly or debilitated patients, severe complications may result from secondary infection. Epidemic or pandemic strains, to which there is little or any natural immunity, may cause fulminate infection even in young and healthy individuals. The only therapeutic agents available are the neuraminidase inhibitors zanamivir (Relenza®; SmithKline Glaxo) and oseltamivir (Tamiflu®; Roche), andamantadine, which is less effective. Consequently control of the disease relies on immunization.

Influenza virus is an orthomyxovirus, and there are three known types. Influenza A causes seasonal, epidemic or pandemic influenza in humans, and may also cause epizootics in birds, pigs and horses. Influenza B and C are associated with sporadic outbreaks, usually among children and young adults. Influenza viruses are divided into strains or subtypes on the basis of antigenic differences in the HA and NA antigens. Each virus is designated by its type (A, B or C), the animal from which the strain was first isolated (designated only if non-human), the place of initial isolation, the strain number, the year of isolation, and the particular HA and NA antigens (designated by H and N respectively, with an identifying numeral).

“Avian influenza” (AI) is caused by influenza A viruses which occur naturally among wild birds, such as ducks, geese and swans. Until an epizootic in Pennsylvania in 1983-84, AI was not regarded as a virulent disease.

“Low pathogenic avian influenza” (LPAI) is common in birds and causes few problems. Wild birds, primarily waterfowl and shorebirds, are the natural reservoir of the low pathogenic strains of the virus (LPAI). Although reservoir birds typically do not develop any clinical signs due to LPAI virus, the virus may cause disease outbreaks in domestic chickens, turkeys and ducks.

“Non-pathogenic avian influenza” is caused by avian influenza virus strains which are able to infect susceptible birds, but does not cause disease symptoms or disease outbreaks.

Highly pathogenic avian influenza” (HPAI) is characterized by sudden onset, severe illness and rapid death of affected birds, and has a mortality rate approaching 100%. HPAI is a virulent and highly contagious viral disease which occurs in poultry and other birds. It was first identified in Italy in the early 1900s. On rare occasions, highly pathogenic avian influenza can spread to humans and other animals, usually following direct contact with infected birds. LPAI and HPAI strains of avian influenza can readily be distinguished by their relative reproduction ratio, infectivity and mortality; HPAI has a significantly higher reproduction ratio, invariably infects susceptible birds such as chickens, and causes death of infected susceptible birds within approximately 6 days after infection. See for example Van der Goot, Koch et al (2003); Van der Goot, de Jong et al (2003).

Only viruses which are of either H5 or H7 subtype are known to be highly pathogenic avian influenza viruses.

These are the two strains of most concern for domestic birds, and for their potential to infect humans. It is thought that HPAI viruses arise from LPAI H5 or H7 viruses infecting chickens and turkeys after spread from free-living birds. At present it is assumed that all H5 and H7 viruses have this potential, and that mutation to virulence is a random event.

For example, influenza virus strain H5N1 is highly pathogenic, deadly to domestic fowl, and can be transmitted from birds to humans. There is no human immunity against HPAI, and no vaccine is available.

Pandemic influenza is virulent human influenza which causes a global outbreak, or pandemic, of serious illness. Influenza A viruses may undergo genetic changes which result in major changes in antigenicity of both the hemagglutinin and the neuraminidase; this is known as antigenic shift. Antigenic shift is thought to result from the fact that influenza A can infect animals as well as humans. A mixed infection, in which strains from different species infect a single host, can lead to reassortment which results in a new influenza virus to which the human population is completely susceptible; an influenza pandemic may result. Because there is little natural immunity, the disease can spread easily from person to person. The most serious influenza pandemics occurred in 1918 (“Spanish flu”), 1957 (“Asian flu”) and 1968 (“Hong Kong flu”). The 1918 influenza pandemic killed approximately 50 to 100 million people worldwide; the 1957 pandemic was responsible for 2 million deaths; and the 1968 outbreak caused about 1 million deaths.

Seasonal or common influenza (interpandemic influenza) is a respiratory illness which can be readily transmitted from person to person. Most people have some immunity, and vaccines are available. These may be live, attenuated vaccines, killed virus (inactivated vaccines), or sub-unit (“split virus”) vaccines. Other types of vaccine are in clinical trial. Small changes in antigenicity of the hemagglutinin or neuraminidase, known as antigenic drift, occur frequently. The population is no longer completely immune to the virus, and seasonal outbreaks of influenza occur. These antigenic changes also require the annual reformulation of influenza vaccines.

A “reassortant” influenza virus is one which has genes derived from more than one influenza virus strain. Usually two influenza virus strains, the Master Donor Virus (MDV) (also known as the master strain, MS) and the strain which is the target for immunization are used. Conventionally, reassortant viruses are obtained by screening viral particles from a mixed viral infection of embryonated eggs or tissue culture host cells. More recently methods of reassortment by reverse genetics have been developed.

Reassortants are conventionally described with reference to the number of genes derived from the respective donor and target viruses. The genes derived from the target virus will usually be the HA and the NA. Thus a 6:2 reassortant has two genes, the HA and the NA genes, from the target virus, and all the other genes from the MS. The 7:1 reassortant according to one embodiment of the first aspect of the present invention has an HA gene of the same type as that of the target highly pathogenic virus, and all the other gens from the MS.

Reassortment, ie the production of reassortants, generally comprises mixing of gene segments from different viruses, usually in eggs or cell culture. Thus conventional annual trivalent vaccines, reflecting the recommended vaccine strains for a particular year, are prepared by the process of 6:2 genetic reassortment. For example, a 6:2 vaccine strain is produced by in vitro co-infection of the relevant A or B strain Master Donor Virus (MDV) with the circulating influenza strain of interest, and antibody-mediated selection of the proper reassortant. The target 6:2 reassortant contains HA and NA genes from the circulating strain, and the remaining genes from the MDV, which is usually selected for high growth in eggs. The reassortant retains the phenotypic properties of the master donor virus. Thus reassortment between two virus types can be used to produce, inter alia, viruses comprising the wild-type epitope strain for one segment, and a cold-adapted attenuated strain for the other segments.

Methods for reassortment of influenza virus strains are well known to those of skill in the art. For example, dilutions of a cold-adapted MDV and a wild-type virus, e.g. a 1:5 dilution of each no matter the concentration of the respective solution, are mixed and then incubated for 24 and 48 hours at 25° C. and 33° C. Reassortment of both influenza A virus and influenza B virus has been used both in cell culture and in eggs to produce reassorted virus strains. See Wareing et al., 2002. Reassortment of influenza strains has also been performed with plasmid constructs. See PCT/US03/12728 filed Apr. 25, 2003, PCT/US05/017734, filed May 20, 2005; and US20050186563.

Unfortunately sometimes large numbers of reassortments need to be performed in order to prepare the desired reassortants. After being reasserted, the viruses can be selected to find the desired reassortants. The desired reassortants can then be cloned to expand their number. Alternatively co-infection of strains, typically into cell culture, can be followed by simultaneous selection and cloning, again typically in cell culture. The reassortment process can be optimized in order to reduce the number of reassortments needed, and thus to increase the throughput or stability of the vaccine production process, etc. Such optimization techniques are typically performed in cell culture, e.g. in CEK cells. See for example International patent application No. PCT/US04/05697 filed Feb. 25, 2004. If a reassortant produces low yields in eggs, it can readily be adapted to growth in this environment by serial passage in eggs, as described for example by Rudneva et al (2007).

A “cross-protective immune response” is one which protects against infection by an influenza virus strain which is not identical to the one used to elicit the response.

An “adjuvant” is a substance which augments, stimulates, activates, potentiates, or modulates the immune response at either the cellular or humoral level.

An adjuvant may be added to a vaccine, or may be administered before administering an antigen, in order to improve the immune response, so that less vaccine is needed to produce the immune response. Widely-used adjuvants include alum, ISCOMs which comprise saponins such as Quil A, liposomes, and agents such as Bacillus Calmette Guerin (BCG), Corynebacterium parvum or mycobacterial peptides which contain bacterial antigens. Other adjuvants include, but are not limited to, the proprietary adjuvant AS04 (GlaxoSmithKline), which is composed of aluminium salt and monophosphoryl lipid A; surfactants, e.g. hexadecylamine, octadecylamine, lysolecithin, di-methyldioctadecylammonium bromide, N,N-dioctadecyl-n′-N-bis(2-hydroxyethylpropane diamine), methoxyhexadecyl-glycerol, and pluronic polyols; polyanions, e.g. pyran, dextran sulphate, polyinosine-cytosine, polyacrylic acid, and carbopol; peptides, e.g. muramyl dipeptide, dimethylglycine, and tuftsin; oil emulsions, and mixtures thereof. Some of these are currently approved for human or veterinary use; others are in clinical trial.

In the description of the invention and in the claims which follow, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

As used herein, the singular forms “a”, “an”, and “the” include the corresponding plural reference unless the context clearly dictates otherwise. Thus, for example, a reference to “an enzyme” includes a plurality of such enzymes, and a reference to “an amino acid” is a reference to one or more amino acids.

Where a range of values is expressed, it will be clearly understood that this range encompasses the upper and lower limits of the range, and all values in between these limits.

### Abbreviations

Abbreviations used herein are as follows:
CA cold-adapted
EID₅₀ fifty percent egg infectious dose
ELISA enzyme-linked immunosorbent assay
HA hemagglutinin
HAI hemagglutinin inhibition
HAU hemagglutinin units
HPAI highly pathogenic avian influenza A
HK/156 influenza virus A/Hong Kong/156/97
HK/213 influenza virus A/Hong Kong/213/03
HK/483 influenza virus A/Hong Kong/483/97
IIV intramuscular inactivated influenza vaccine
i.n. intranasal
i.m. intramuscular
i.v. intravenous
LAIV live attenuated influenza vaccine
LD₅₀ fifty percent lethal dose
LIV live influenza vaccine
LPAI low pathogenic avian influenza A
Len17 influenza virus A/Leningrad/134/17/57
MID₅₀ fifty percent mouse infective dose
MS master strain
MDV master donor virus
NA neuraminidase
PBS phosphate-buffered saline
Pot/86 influenza virus A/Duck/Potsdam/1402-6/86
PCR polymerase chain reaction
p.i. post-infection
ts temperature-sensitive

It is to be clearly understood that this invention is not limited to the particular materials and methods described herein, as these may vary. It is also to be understood that the terminology used herein is for the purpose of describing particular embodiments only, and it is not intended to limit the scope of the present invention, which will be limited only by the appended claims.

Unless defined otherwise, all technical and scientific terms used herein have the same meaning as commonly understood by one of ordinary skill in the art to which this invention belongs. Although any materials and methods similar or equivalent to those described herein can be used to practise or test the present invention, the preferred materials and methods are described.

Wild type H5N1 viruses used in this study were A/Hong Kong/156/97 (HK/156), A/Hong Kong/483/97 (HK/483), and A/Hong Kong/213/03 (HK/213).

Viruses were propagated in the allantoic cavity of 10-day-old embryonated hens' eggs at 34° C. for 2 days (Len17/H5, Len17, and Pot/86) or at 37° C. for 26-28 h (HK/156, HK/1483, and HK/213). Allantoic fluid was collected after 26 h (H5N1 viruses) or 48 h (Len17/H5 and Len17) post-inoculation. Virus stocks were aliquoted and stored at −70° C. until use. Fifty percent egg infectious dose (EID₅₀) titres were determined by serial titration of virus in eggs and calculated by the method of Reed and Muench (1938).

The invention will now be described in detail by way of reference only to the following non-limiting examples and drawings.

### EXAMPLE 1

### Preparation of Reassortant Strain

A strategy similar to that used for production of reassortant strains derived from the attenuation donor A/Leningrad/134/17/57(H2N2) with the contemporary epidemic viruses H1N1 and H3N2 was used for the development of a vaccine strain comprising surface antigens of the avian influenza virus sub-type H5N2.

A/17/Duck/Potsdam/86/92(H5N2) was obtained by the method of classical genetic reassortment of the non-pathogenic avian virus A/17/Duck/Potsdam/1402-6/86(H5N2) with the cold-adapted, temperature sensitive master donor strain A/Leningrad/134/17/57(H2N2) in developing chick embryos, with subsequent selection against the A/Leningrad/134/17/57(H2N2) attenuation donor strain in the presence of anti-serum against the attenuation donor strain.

The genome of the reassortant strain was analysed by PCR restriction analysis (Klimov A. I., Cox N. J.: J. Virol. Method. 1995. No. 55. p. 445-446), and partial or complete DNA sequencing of separate genes was carried out. This demonstrated that the reassortant A/17/Duck/Potsdam/86/92(H5N2) inherited its HA gene from a parent avian virus of sub-type H5N2, while the NA gene and six genes encoding non-glycolysated proteins were inherited from the A/Leningrad/134/17/57(H2N2) attenuation donor. Thus this is a 7:1 reassortant, in contrast to conventional vaccine strains, which are 6:2 reassortants. The reassortant was designated Len17/H5.

The hemagglutinin inhibition reaction (HAI) was used to confirm that the hemagglutinin type of the reassortant was the same as that of the parent strain, wild-type A/Duck/Potsdam/1402-6/86(H5N2). The strain is temperature-sensitive (difference in titre is 6.8 logEID₅₀/ml at 33° C. and 40° C.) and cold-adapted (difference in titre is 3.1 logEID₅₀/ml at 33° C. and 25° C.).

Therefore the A/17/Duck/Potsdam/86/92(H5N2) vaccine strain according to the invention has a combination of useful properties which are necessary for a vaccine strain:

(a) the antigenic specificity of the wild type A/Duck/Potsdam/1402-6/86(H5N2) virus hemagglutinin;
(b) the genome structure required for reassortant vaccine strains;
(c) temperature-sensitivity and cold-adaptation, which is correlated with the attenuation which is typical for the master donor strain.

A sample of the reassortant strain has been deposited in the Russian State Collection of Viruses on 10 Feb. 2006 under Accession No. 2389. The strain morphology was polymorphous, which is typical of influenza viruses.

### EXAMPLE 2

### Evaluation of the Reassortant Strain

Infectious activity, as assessed by replication in developing chicken embryos incubated at 33° C. for 48 hours, was 9.3 logEID₅₀/ml.

The hemagglutinin titre was 1:512.
Genetic stability of the biological features of the strain was demonstrated after intra-nasal passage in ferrets.
The characteristics of the reassortant strain A/17/Duck/Potsdam/86/92(H5N2) are summarized below.
1. Strain name: A/17/Duck/Potsdam/86/92(H5N2)
2. Series: Series 1.
3. Method of production: re-assortment;

parental viruses:

A/Duck/Potsdam/1402-6/86(H5N2) epidemic virus
A/Leningrad/134/17/57(H2N2) attenuation donor
4. Number of passages in the recombination process: 7
5. Characteristics of the strain before lyophilisation: Optimum incubation conditions for production: 33° C., 48 hours;
Haemagglutinin activity 1:512;
Infectious activity 8.5±0.3 logEID₅₀/0.2 ml;
Sensitivity to serum inhibitors: inhibitor-resistant
Difference in infectious activity at 33° C. and 40° C., 6.8 logEID₅₀/ml;
Difference in infectious activity at 33° C. and 25° C.: 3.1 lgEID₅₀/ml;
Genome structure of the reassortant:
Genes from non-pathogenic avian influenza: HA
Genes from attenuation donor: PA, PB1, PB2, NP, M, NS, NA
6. Characteristics of the strain after lyophilisation:
Lyophilisation date: 24 Nov. 2005;
Amount of material per flask: 1 ml;
Number of doses in series: 4.
Infectious activity: 7.5 logEID₅₀/0.2 ml;
Haemagglutinin titre: 1:256.
7. Recommended dilution at vaccination 1:2
8. Antigenic specificity:
Haemagglutinin: identical to A/17/Duck/Potsdam/86/92(H5N2) virus as assessed by HAI with rat anti-serum.
Neuraminidase: identical to A/Leningrad/134/17/57(H2N2) virus as assessed by sequencing.
9. Safety for mice following subcutaneous or intranasal administration: harmless.
10. Bacteriological control of lyophilised material: date—30 Nov. 2005: sterile.
11. Control for extraneous viruses: no extraneous viruses.

### EXAMPLE 3

### Pathogenicity of the Candidate Vaccine Strain in Chickens

The effects of intranasal and intravenous administration of A/17/Duck/Potsdam/86/92(H5N2) strain to chickens were assessed. Intravenous administration of 0.2 ml vaccine virus proved to be harmless, and no symptoms of disease were observed in any of the eight birds tested. Following intranasal administration of 0.5 ml vaccine virus, no symptoms of disease were observed. The virus was not excreted from oropharyngeal and cloacal swabs, and did not induce seroconversion in any of the five birds tested, as shown in Table 1.

Thus reassortant A/17/Duck/Potsdam/86/92(H5N2) demonstrated a high level of attenuation for chickens, which indicated that this strain was safe for vaccine manufacture in eggs and use in chickens.

TABLE 1
Safety of reassortant strain A/17/Duck/Potsdam/86/92
(H5N2) following administration to chickens

| | | | Safety and adaptation | | | | |
|---|---|---|---|---|---|---|---|
| | | | on intranasal administration | | | | |
| | Non-pathogenicity | | | | Virus excretion on day | | |
| | on intravenous | | | | 3 after infection | | |
| | administration | | | | Oropharyngeal | Cloacal | Serum |
| Viruses | Morbidity | Mortality | Morbidity | Mortality | swabs | swabs | conversions |
| A/17/Duck/Potsdam/86/92(H5N2) | 0/8 | 0/8 | 0/5 | 0/5 | 0/5 | 0/5 | 0/5 |
| Vaccine strain | | | | | | | |
| A/Duck/Potsdam/1402-6/86(H5N2) | 0/8 | 0/8 | 0/5 | 0/5 | 0/5 | 0/5 | 3/5 |
| Wild-type virus | | | | | | | |

### EXAMPLE 4

### Pathogenicity and Protective Effect of the Candidate Vaccine Strain in Mice

Strain A/17/Duck/Potsdam/86/92(H5N2) is safe, immunogenic and effective against subsequent infection with highly pathogenic virus of sub-type H5N1 on intranasal administration to mice.

The safety and immunogenicity of reassortant strain A/17/Duck/Potsdam/86/92(H5N2) was studied in BALB/c mice, using intranasal administration of 6-7 log EID₅₀. The reassortant was attenuated for mice, reproducing more effectively in nasal passages (3.5 log EID₅₀/ml) than in lung tissue (1.8 log EID₅₀/ml), as shown in Table 2.

TABLE 2
Safety of reassortant strain A/17/Duck/Potsdam/86/92(H5N2) following intranasal
administration to mice

| | | Replication | | | |
|---|---|---|---|---|---|
| | Replication | in nasal | | | |
| | in lungs | passages | | | |
| | (log | (log | | Maximum weight | |
| Viruses | EID₅₀/ml) | EID₅₀/ml) | logMID₅₀ | loss (%) | logLD₅₀ |
| A/17/Duck/Potsdam/86/92 | 2.1 ± .0 | 3.5 ± 0.0 | 7.0 | 1 | >7.0 |
| (H5N2) | | | | | |
| Vaccine strain | | | | | |
| A/Duck/Potsdam/1402-6/86 | 6.3 ± 0.3 | 1.6 ± 0.2 | 3.3 | 4 | >7.0 |
| (H5N2) | | | | | |
| Wild-type virus | | | | | |

The humoral immune response in the serum of the experimental animals was assessed at 28 days after the preparations were administered. Using immunoenzyme assay, the presence of specific IgG and IgA against viruses of subtype H5N1, against HK/213 whole virus and against purified recombinant HA of HK/483 virus was detected.

These results are summarised in Table 3.

TABLE 3
IgG and IgA specific antibodies to H5N1 viruses in serum
of mice immunised with reassortant virus
A//17/Duck/Potsdam/86/92 (H5N2)

| | |
|---|---|
| IgG | IgA |

| Viruses | HK/213 | HK/483 | HK/213 | HK/483 |
|---|---|---|---|---|
| Len17/H5 | 4.7 | 3.2 | 2.8 | 2.4 |
| H5N2-DT | 5.1 | 4.5 | 3.1 | 2.6 |
| Control | <2.0 | <2.0 | <2.0 | <2.0 |

Mice immunised with a single dose of 300 MID₅₀ of LIV prepared from A/17/Duck/Potsdam/86/92(H5N2) strain were 100% protected from lethal infection by HK/483 virus at a dose of 50 LD₅₀, while 100% mortality was observed in a control group of animals. A single intranasal immunisation with LIV resulted in 100% protection against subsequent challenge with 100 MID₅₀ HK/213 virus, and no infectious virus was extracted from the lungs of any of the five test animals. These results are summarised in Table 4.

TABLE 4
Resistance of mice immunised with reassortant virus
A/17/Duck/Potsdam/86/92(H5N2) to infection with viruses of
sub-type H5N1

| | |
|---|---|
| | Infection |
| Infection | 100 MID₅₀ |
| 10 LD₅₀ | A/Hong |
| A/Hong Kong/483/97 | Kong/213/2003 |

| | | Maximum | Mean virus titres |
|---|---|---|---|
| | | weight loss | from lung tissue |
| Viruses | Mortality | (%) | (log EID₅₀/ml) |
| Len17/H5 | 0/8 | 5 | <1.5 |
| H5N2-DT | 0/8 | 0 | <1.5 |
| Control | 0/8 | 23 | 4.9 |

Thus we have shown that LIV from a reassortant vaccine strain containing HA from the non-pathogenic avian virus H5N2 was safe and immunogenic, and that a single administration of the vaccine elicited a protective immune response against subsequent challenge with highly pathogenic viruses of sub-type H5N1, including viruses significantly different in their antigenic properties from those of the immunizing strain.

### EXAMPLE 5

### Comparison of the immunogenic properties of LAIV and IIV from A/17/Duck/Potsdam/86/92(H5N2) in Mice

An examination of the infectious properties of whole-virion vaccine prepared using the 17/H5 strain with the addition of aluminium hydroxide adjuvant demonstrated that a single dose of vaccine without adjuvant was not sufficient to protect against subsequent infection with the highly pathogenic H5N1 virus found in 2005 in Vietnam. To provide 100% protection against infection with this virus, two doses of the vaccine with adjuvant were required. These results are discussed in more details in examples 11 and 13, and are summarised in Tables 9 and 11.

### EXAMPLE 6

### Production of an Immunogenic Reassortant of Avian Influenza Virus H7N1

The outbreak of highly pathogenic avian influenza H7N1 which emerged in The Netherlands during 2003 resulted in more than 80 human cases of conjunctivitis and mild respiratory illnesses, and 1 fatal case. To prepare candidate live influenza vaccines for protection against a potential future pandemic we used genetic reassortment with non-pathogenic avian viruses and the cold-adapted H2N2 master strain A/Leningrad/134/17/57 (Len/17). Len/17 is currently used in Russia for preparing approved live attenuated vaccines for adults and children. We showed in Examples 3 and 4 that a reassortant between Len/17 and non-pathogenic H5 influenza virus is attenuated for chickens and mice.

We evaluated a 6:2 reassortant between influenza virus Len/17 and influenza virus A/Mallard/Netherlands/12/00(H7N3). The reassortant A/17/Mallard/Netherlands/00/84(H7N3) (Len17/H7) demonstrated cold-adapted (ca) and temperature-sensitive (ts) phenotypes similar to those of Len/17. The HA gene sequence of Len17/H7 was identical to that of the parent H7N3 wild type virus. The results of a hemagglutination inhibition (HI) test with a panel of ferret antisera to different avian and human H7 viruses showed that the antigenic profile of the reassortant was similar to that of the H7N3 wild type parent strain as well as to that of human H7 isolates from the Netherlands, including the virus isolated from the fatal case.

The reassortant demonstrated high growth capacity in embryonated chicken eggs at optimal temperature (34° C.), comparable to that of the parent Len/17 MS. Moreover Len17/H7 was shown to be attenuated for chickens, like the Len/17 parent, whereas H7N3 wild type virus caused 60% mortality. Like the Len/17 master strain, Len17/H7 was completely attenuated for mice.

After intranasal inoculation with 105-10⁶ EID₅₀, Len17/H7 replicated well in nasal passages of mice, but did not replicate in mouse lung. Despite the lack of replication in mouse lung, Len17/H7 induced serum virus-specific IgG titres as high as 3.7±0.8 log₁₀.

### EXAMPLE 7

### Evaluation of Immunogenicity

We compared the immunogenicity and protective efficacy of an LAIV prepared from a H5N2 reassortant and two types of inactivated subunit vaccines used in Russia. Both subunit vaccines were prepared from a H5N1 strain using reverse genetics methods. One of the subunit vaccines also contained a polymer adjuvant, Polyoxidonium (copolymer of N-oxidized 1,4-ethylenepiperazine and (N-carboxyethyl) 1,4-ethylenepiperazine bromide, molecular weight 100 kDa; Petrovax Pharm, Moscow, Russia), while the other contained alum adjuvant.

Table 5 shows the results obtained after challenge with highly pathogenic H5N1 virus. It is evident that the LAIV evokes a very high level of cross-protection, and this was 570-87% after the first and second doses.

TABLE 5
Evaluation of immunogenicity and protection in mice after
immunization with H5 vaccine candidates

| | | | | | % survivals after |
|---|---|---|---|---|---|
| | | | | | challenge with 27 |
| Number of | | Titres after vaccination | | | LD₅₀ of |
| doses | Vaccine | ELISA | Neutralisation | HAI | A/Ch/Kurgan/02/05 |
| 1 | Subunit vaccine | <1:40 | <1:10 | <1:10 | 20 |
| | H5N1 + polymer* | | | | |
| | Subunit vaccine | 1:80 | 1:10 | <1:10 | 40 |
| | H5N1 + Alum* | | | | |
| | LIV H5N2 | 1:320 | 1:14 | 1:40 | 57 |
| 2 | Subunit vaccine | 1:80 | 1:20 | 1:20 | 50 |
| | H5N1 + polymer** | | | | |
| | Subunit vaccine | 1:320 | 1:20 | 1:10 | 80 |
| | H5N1 + Alum** | | | | |
| | LIV H5N2*** | 1:1280 | 1:40 | 1:80 | 87 |
| Control | Control | <1:40 | <1:10 | <1:10 | 0 |

*H5N1 vaccines containing 15 μg HA were generated from the NIBRG-14 strain, obtained from NIBSC UK;
**2 doses at an interval of 21 days
***2 doses of 106.4 EID50/0.05 ml at an interval of 10 days

### EXAMPLE 8

### Pathogenicity and Infectivity in Chickens

For the determination of pathogenicity, eight chickens per group were inoculated intravenously (i.v.) with 0.2 ml of a 10⁻¹ dilution of each virus, and observed daily for 14 days for clinical signs and death. To determine infectivity, five chickens were inoculated intranasally (i.n.) with 10⁶ EID₅₀ of each virus in 0.1 ml. On day 3 post-inoculation (p.i.), oropharyngeal and cloacal swabs were collected from each chicken and virus replication was assessed in embryonated chicken eggs. The chickens were observed for clinical signs of disease and death for 21 days, at which time serum samples were harvested and tested for the presence of antibodies by the agar gel immunodiffusion (AGID) test.

The two parent and reassortant Len17/H5 viruses were administered to specific pathogen free (SPF) chickens to determine their potential risk for animal production. This included assessment of the ability to cause morbidity and mortality following i.v. inoculation (pathogenicity) and the level of tissue-specific replication following simulated natural exposure (i.n. inoculation). With i.v. or i.n. inoculation, no clinical disease signs or deaths were observed in the chickens with any of the three viruses over the 14 or 21 days observation period, respectively, as shown in Table 6. For the i.n. inoculated group on day 3 p.i., which is the peak replication time for low pathogenic avian influenza viruses, virus was not isolated from respiratory (oropharyngeal swab) or intestinal (cloacal swab) tracts, but antibodies to avian influenza viral proteins were detected in chickens inoculated with the avian Pot/86 parent virus.

TABLE 6
Pathogenicity and infectivity of the reassortant Len17/H5 and parent viruses in chickens

i.n. pathogenicity and infectivity^{b}

| | i.v. pathogenicity^{a} | | | | Virus | | |
|---|---|---|---|---|---|---|---|
| | Morbidity | Mortality | Morbidity | Mortality | detection in swabs | | Seroconveresion |
| Viruses | (sick/total) | (dead/total) | (sick/total) | (dead/total) | Oropharengeal | Cloacal | (AGID) |
| Len17/H5 | 0/8 | 0/8 | 0/5 | 0/5 | 0/5 | 0/5 | 0/5 |
| Len17 | 0/8 | 0/8 | 0/5 | 0/5 | 0/5 | 0/5 | 0/5 |
| Pot/86 | 0/8 | 0/8 | 0/5 | 0/5 | 0/5 | 0/5 | 3/5 |

^{a}Groups of eight chickens were infected i.v. with 0.2 ml 1:10 dilution of each virus and observed daily for 14 days for clinical signs and death
^{b}Groups of five chickens were infected i.n. with 0.1 ml of 10⁶ EID₅₀ of each virus. The oropharyngeal and cloacal swabs were collected 3 days p.i. and titrated in eggs for assessing viral replication. The chickens were observed for clinical signs of disease and death for 21 days. To determine infectivity, sera were collected 21 days p.i. and tested for the presence of antibodies by agar gel immunodiffusion (AGID) test.

The combined data from the two experiments suggests that the two parent and reassortant Len17/H5 viruses were not highly pathogenic for chickens. Following simulated natural exposure, the Pot/86 parent virus apparently replicated poorly in chickens; evidence of infection was only detected by the presence of antibodies, and not by actual detection of virus in the respiratory or intestinal tracts. A similar resistance to infection has been reported following inoculation of chickens with viruses isolated from wild waterfowl (Jones and Swayne, 2004). Furthermore, reassortant Len17/H5 failed to replicate in chickens following simulated natural exposure by i.n. inoculation. These observations suggest that the use of the reassortant Len17/H5 in manufacturing human vaccines will not pose a threat to the poultry industry.

### EXAMPLE 9

### Pathogenicity and Infectivity of the Vaccine in Mice

Ten-week-old female BALB/c mice (Jackson Laboratories, Bar Harbor, Me., USA) were lightly anesthetized with CO₂, and 50 μl of 10¹ to 10⁷ EID₅₀ of Len17/H5, Len17, or Pot/86 diluted in phosphate-buffered saline (PBS) was inoculated i.n. A 50% mouse infectious dose (MID₅₀) was used to determine infectivity and a 50% lethal dose (LD₅₀) was used to determine pathogenicity, as described by Lu et al (1999). To evaluate the replication of Len17/H5 and the two parent viruses, mice were infected i.n. with 106 EID₅₀ of these viruses. Organ samples were collected on day 3 (lung and nose) and day 6 (brain) p.i. and titrated for infectious virus in eggs.

As shown in Table 7, reassortant Len17/H5 and two parent viruses were all non-lethal for mice (LD₅₀>10⁷ EID₅₀). Like the Len17 Ca H2N2 donor strain, Len17/H5 virus had 10-fold higher MID50 compared with the parent Pot/86 virus. Replication of the reassortant Len17/H5 virus in the upper and lower respiratory tract of mice was evaluated as a measure of attenuation. Mice were infected i.n. with 10⁶ EID₅₀ of the parent and reassortant viruses, and the titres of virus present in the nose and lungs were determined 3 days p.i.

TABLE 7
Pathogenicity, infectivity and replication of the reassortant Len17/H5
and parent viruses in mice

| | | | | | | Number of | |
|---|---|---|---|---|---|---|---|
| | Pathogenicity & | | | | | infected/total | |
| | infectivity^{a} | | Maximum mean | Mean virus titres^{c} | | number^{c} | |
| Viruses^{a} | MID₅₀ | LD₅₀ | weight loss (%)^{b} | Lung | Nose | Lung | Nose |
| Len17/H5 | 4.3 | >7 | 1 | 2.1 ± 1.0 | 3.5 ± 0.0 | 1/3 | 3/3 |
| Len17 | 4.8 | >7 | 1 | 2.3 ± 0.5 | 2.7 ± 0.2 | 3/3 | 3/3 |
| Pot/86 | 3.3 | >7 | 4 | 6.3 ± 0.3 | 1.6 ± 0.2 | 3/3 | 1/3 |

^{a}Mice were infected i.n. with 10¹ to 10⁷ EID₅₀ of each virus. Three days later, three mice from each dilution were euthanized; lung and nose were collected and titrated for virus infectivity in eggs. The five remaining mice in each dilution were checked daily for disease signs, weight loss and death for 14 days p/i. Lung virus titres were used for the determination of MID₅₀ of Pot/86 and nose virus titres were used for the determination of MID₅₀ of Len17/H5 and Len17 viruses. MID₅₀ and LD₅₀ are expressed as the log₁₀EID₅₀ required to give one MID₅₀ or one LD₅₀.
^{b}Maximum mean weight loss (%) was determined in the group of mice infected i.n. with 10⁶ EID₅₀ of each virus.
^{c}Mice were infected i.n. with 10⁶ EID₅₀ of each virus. Lung and nose tissues were collected on 3 days p.i. and titrated in eggs for assessing viral replication. The virus titres are expressed as the mean log₁₀ EID₅₀/ml ± S.D. from three mice per group. The limit of virus detection was 10^{1.5} EID₅₀/ml. Tissues in which no virus was detected were given a value of 10^{1.5} EID₅₀ /ml for calculation of the mean titre. Mice were considered infected if virus was detected in 0.1 ml of 1:10 dilution of tissue homogenate.

The parental Pot/86 virus replicated efficiently in mouse lungs, but poorly in the nose. In contrast, the Len17/H5 reassortant replicated well in the nose but poorly in the lungs, as did the Len17 ca strain for which virus was recovered from only one of three mouse lungs (titre=10^{3.3} EID₅₀/ml). None of the viruses were detected in the brains of any infected mouse on day 6 p.i. (data not shown). These results indicated that the reassortant Len17/H5 virus replicated predominantly in the upper respiratory tract and was attenuated in mice.

### EXAMPLE 10

### Immunogenicity and Cross-Reactive Antibody Response in Mice

Next, we evaluated the immunogenicity of Len17/H5 inoculated in. as an LAIV at a single dose of 300 MID₅₀ or i.m. as an IIV (one dose of 10 μg whole virus) in mice.

The high-growth Len17/H5 virus was concentrated from allantoic fluid and purified on a sucrose gradient using the method of Cox et al (1984), and prepared as IIV by treating purified virus with 0.025% formalin at 4° C. for 3 days. A group of mice was injected intramuscularly (i.m.) with one dose of 10 μg of IIV (˜3 μg HA protein) in a volume of 0.1 ml.

Mice were inoculated i.n. with one dose of 300 MID₅₀ (˜10⁷ EID₅₀) of LAIV or injected intramuscularly (i.m.) with one or two doses of 10 μg (˜3 μg of HA protein) of IIV, with or without alum adjuvant (Li et al, 1999). Some mice received two inoculations at an interval of 4 weeks.

Groups of 8-week-old female BALD/c mice were immunized i.n. with one dose of 300 MID₅₀ of Len17/H5 (=107 EID₅₀) or Len17 (=10^{7.3} EID₅₀) LAIV. Mice were infected i.n. with either 300 MID₅₀ of Pot/86 (=10^{5.7} EID₅₀) or 100 MID₅₀ of HK/2I3 virus (=10^{3.8} EID₅₀) as positive controls, and received PBS as a negative control.

Six weeks after i.n. or i.m. immunization, blood, lung and nasal wash samples were collected from five mice per group, as previously described (Katz et al, 1997). Sera were treated with receptor-destroying enzyme (neuraminidase) from Vibrio cholerae (Denka-Seiken, Tokyo, Japan) before testing for the presence of H5-specific antibodies (Kendal et al, 1982). Titres of neutralizing antibody were determined using a microneutralization assay as previously described (Rowe et al, 1999). Neutralizing antibody titres are expressed as the reciprocal of the highest dilution of serum that gave 50% neutralization of 100 TCID₅₀ of virus in Madin-Darby Canine Kidney cells.

Influenza H5-specific IgG and IgA antibodies were detected by an enzyme-linked immunosorbent assay (ELISA) as previously described (Katz et al, 1997), except that 2 μg/ml of a purified baculovirus-expressed H5 (HK/156) recombinant HA protein (Protein Sciences Corporation, Meriden, Conn., USA) was used to coat the plates. The end-point ELISA titres were expressed as the highest dilution which yielded an optical density (CD) greater than twice the mean CD plus standard deviation (S.D.) of similarly diluted control samples.

Six weeks after immunization, sera, lung and nasal washes were collected and tested for H5 virus-specific antibodies by microneutralization assay or ELISA (Katz et al, 1997; Rowe et al, 1999). As shown in Table 8, neutralizing antibodies against the homologous Pot/86 virus were detected in serum of mice receiving LAIV Len17/H5, but cross-reactive neutralizing antibodies against HPAI H5N1 HK/156 or HK/213 virus were not detected.

TABLE 8
Neutralizing antibody responses of mice immunized i.n. or
i.m. with H5 influenza vaccines

| | |
|---|---|
| Vaccine group | Neutralizing antibody titre^{b} against |

| (route)^{a} | Pot/86 | Len17 | HK/156^{c} | HK/213 |
|---|---|---|---|---|
| Len17/H5 (i.n.) | 80 | 20 | 20 | 20 |
| Len17/H5 (i.m.) | 160 | 20 | 80 | 20 |
| Pot/86 (i.n.) | 160 | 20 | 80 | 40 |
| Len17 (i.n.) | 20 | 160 | 20 | 20 |
| HK/213 (i.n.) | 20 | 20 | 20 | 640 |
| PBS (i.n.) | 20 | 20 | 20 | 20 |

Values in italics represent titres to the homologous virus.
^{a}BALB/c mice were either infected i.n. with one dose of 300 MID₅₀ of LAIV or injected i.m. with one dose of 10 μg of Len17/H5 IIV. Two groups of mice were infected i.n. with either 300 MID₅₀ of Pot/86 wild-type virus or 100 MID₅₀ of HK/213 virus as positive controls. Another group of mice received PBS as a negative control.
^{b}Sera were collected 6 weeks after vaccination or infection and pooled from five mice per group to test pre-challenge neutralizing antibodies against H5 and H2 viruses.
^{c}Antigenically related HK/156 virus was used instead of the challenge virus HK/483 because the latter virus is less sensitive in the microneutralization assay (data not shown).

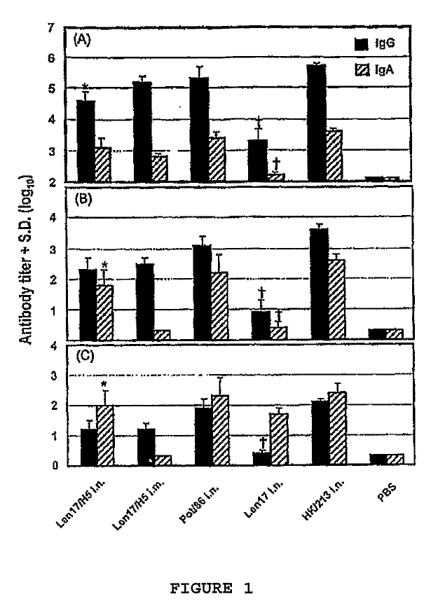

However, as shown in FIG. 1, substantial levels of H5N1 virus-specific serum IgG and respiratory tract IgA were detected by ELISA. As expected, the Len17 ca H2N2 parent virus did not induce any detectable cross-reactive neutralizing antibodies against the H5 viruses, but a low level of serum IgG cross-reactive with H5 HA which was 20- to 100-fold less (p<0.01) than the subtype-specific IgG response induced by Len17/H5 as a live or killed vaccine, respectively, was detected.

Interestingly, FIG. 1 also shows that the Len17 ca parent virus induced titres of H5-cross-reactive nasal IgA which were not significantly different to those induced by Len17/H5 LAIV, suggesting that the local IgA response was generally more subtype-cross-reactive than the serum IgG antibody response. When used as a formalin-inactivated vaccine, Len17/H5 elicited similar neutralizing antibody titres (160 and 80, respectively) to the homologous Pot/86 virus and antigenically related HK/156 virus, but neutralizing antibodies which cross-reacted with HK/2l3 virus were not detected (Table 8).

As shown in FIG. 1, the inactivated Len17/H5 vaccine also induced significant levels of HK/156 HA-specific IgG in serum, lung and nasal washes. The IgA and/or IgG antibodies which cross-reacted with HK/213 virus in serum, lung and nasal washes were also observed in mice receiving either Len17/H5 LAIV or Len17/H5 IIV.

In summary, IIV inoculated by the i.m. route induced better cross-reactive serum neutralizing and IgG (p<0.05) antibody responses to HK/156 virus HA compared to the LAIV Len17/H5, while the latter vaccine induced superior H5 HA-specific IgA antibody responses in respiratory tract washes.

### EXAMPLE 11

### Cross-Protective Efficacy of the Reassortant Len17/H5 Vaccine in Mice

The protective efficacy of Len17/H5 as an LAIV or IIV was evaluated in mice challenged with H5N1 viruses isolated from humans in Hong Kong in 1997 (HK/483) and 2003 (HK/213). HK/483 was chosen to represent the 1997 H5N1 viruses, since it had previously been shown to be highly lethal for naive BALB/c mice (Lu et al, 1999). The antigenically variant H5N1 virus, HK/213, was not lethal for mice, but replicated to high titres in mouse lungs.

Six weeks after i.n. or i.m. immunization, vaccinated mice were challenged i.n. with 50 μl of 100 MID₅₀ of HK/2I3 or 50 LD₅₀ of HK/483. Three or 6 days after challenge, five animals per group were euthanized and the tissues were collected and stored at −70° C. Thawed tissues were homogenized in 1 ml of cold PBS and titrated for virus infectivity in 1-day-old embryonated eggs, as previously described (Lu et al, 1999). Virus endpoint titres were expressed as the mean log₁₀EID₅₀/ml±S.D. The eight mice in each group which were challenged i.n. with the highly pathogenic HK/483 virus were observed daily for signs of disease, weight loss and death for 14 days after challenge. The statistical significance of the results was determined using the two-tailed Student's t-test.

In the first experiment, groups of vaccinated mice (n=13) were infected i.n. with 50 LD₅₀ of HP HK/483. Eight mice per group were monitored daily for weight loss and death for 14 days. The remaining mice in each group were euthanized on day 6 p.i. to determine the levels of viral replication in the lower (lung) and upper (nose) respiratory tract, brain, and thymus. Day 6 was chosen to evaluate cross-protection, because we have found that naive mice have substantial titres of HK/483 virus in lung and nose, and have peak of viral replication in brain and thymus at this time point. The results are summarized in Table 9.

TABLE 9
Protective efficacy of H5 influenza vaccines against infections
with 1997 and 2003 H5N1 viruses

| | Challenge with HK/483^{b} | | | | | | Challenge with HK/213^{c} | |
|---|---|---|---|---|---|---|---|---|
| | Maximum | Number of | | | | | Mean virus | Number of |
| Vaccine group | weight | death/total | Mean virus titres | | | | titre | protected/total |
| (route)^{a} | loss (%) | number | Lung | Nose | Brain | Thymus | (lung) | number |
| Len17/H5 (i.n.) | 6^{d} | 0/8 | 1.9 ± 0.5^{d} | ≦0.8^{d} | ≦0.8^{d} | ≦0.8^{d} | 1.8 ± 0.9^{d} | 9/10 |
| Len17/H5 (i.m.) | 7^{d} | 1/8 | ≦1.5^{d} | 1.1 ± 0.6^{d} | 1.0 ± 0.5^{d} | ≦0.8^{d} | ≦1.5^{d} | 5/5 |
| Pot/86 (i.n.) | 0^{d} | 0/8 | ≦1.5^{d} | ≦0.8^{d} | ≦0.8^{d} | ≦0.8^{d} | ≦1.5^{d} | 10/10 |
| Len17 (i.n.) | 19 | 6/8 | 4.4 ± 1.7 | ≦0.8^{d} | 2.3 ± 0.6^{d} | 1.1 ± 0.1^{d} | 4.7 ± 1.5 | 0/10 |
| PBS (i.n.) | 22 | 8/8 | 5.9 ± 1.3 | 4.0 ± 0.7 | 4.3 ± 0.8 | 3.6 ± 1.3 | 5.3 ± 1.4 | 0/10 |

^{a}BALB/c mice were infected i.n. with one dose of 300 MID₅₀ of LAIV or injected i.m. with one dose of 10 μg of Len17/H5 IIV. Mice were infected i.n. with 300 MID₅₀ of Pot/86 wild-type virus as a positive control or received PBS as a negative control.
^{b}Mice (n = 13/group) were challenged i.n. 6 weeks later with 50 LD₅₀ (=1000 MID₅₀) of HK/483 virus and eight mice per group were observed daily for weight loss and death for 14 days. Virus titres were determined on day 6 p.i. and represent means log₁₀ EID₅₀ ± S.D. of five mice per group. The limit of virus detection was 10^{1.5} EID₅₀/ml for lungs and 10^{0.8} EID₅₀/ml for other organs. Tissues in which no virus was detected were given a value of 10^{1.5} EID₅₀/ml (lung) or 10^{0.8} EID₅₀/ml (other tissues) for calculation of the mean titre.
^{c}Mice (n = 5-10/group) were challenged i.n. 6 weeks later with 100 MID₅₀ of HK/213 virus. Mean lung virus titres and protection from infection were determined on day 3 p.i. Titres represent means log₁₀EID₅₀ ± S.D. of five mice per group. The limit of virus detection was 10^{1.5} EID₅₀/ml for lungs.
^{d}p < 0.01 compared with PBS group.

All unvaccinated control mice which received PBS died 5-9 days after a challenge with HK/483, having a mean maximum weight loss of 22% and high titres of virus in the lung, nose, brain, and thymus on day 6 p.i. In contrast, mice which were inoculated i.n. with the wild-type parental Pot/86 virus exhibited no disease signs over the entire experimental period, and no virus was detected in any organ on day 6 p.i. Mice receiving the ca parent Len17 (H2N2) virus showed severe disease, with a mean maximum weight loss of 19%, but demonstrated a modest increase in survival compared with the unvaccinated group. Consistent with this observation was a modest, but not significant reduction in HK/483 lung viral titres in these mice. On the other hand, viral titres in the upper respiratory tract, brain and thymus were significantly lower in mice which received the parent Len17 (H2N2) virus compared with those which received PBS only. Similar heterosubtypic protection against H5N1 viruses has been observed previously (Tumpey et al, 2001).

In contrast, all mice receiving the Len17/H5 LAIV survived a lethal challenge with HP HK/483 virus, but exhibited mild disease, as measured by a modest weight loss observed between day 3 and 5 p.i. (data not shown). Only low titres of virus were detected in lungs of two of five Len17/H5 LAIV vaccinated mice (10^{2.3} and 10^{2.5} EID₅₀/ml) on day 6 p.i., and no virus was detected in any other organs tested, indicating that these mice were effectively protected from the HP HK/483 challenge. When delivered as an IIV, Len17/H5 protected seven of eight mice from lethal HK/483 virus disease, although the mice experienced modest weight loss. While no virus was detected in the lungs or thymus of mice vaccinated with Len17/H5 IIV, low titres of virus were isolated from the nose of one of five mice (10¹⁶ EID₅₀/ml), and the brains of two of five mice (10^{1.6} and 10^{1.8} EID₅₀/ml) on day 6 p.i.

In a second experiment, five mice in each vaccine group were challenged i.n. with 100 MID₅₀ of HK/2I3 2003 virus and viral lung titres on day 3 p.i. were determined. Mice given only PBS had high titres of virus in the lungs on day 3 p.i. The lung viral titres in the Len17-immunized mice were slightly lower than those of unvaccinated PBS mice but the difference was not significant. As observed with the HK/483 challenge, no virus was detected in the lungs of any mouse inoculated with the wild-type parental Pot/86 H5N2 virus 3 days after challenge with HK/213 virus. Nine of 10 mice receiving the Len17/H5 LAIV and all mice receiving Len17/H5 IIV lacked detectable HK/213 virus in the lungs on day 3 p.i., which represented at least a 3000-fold reduction in titre compared with the mice receiving PBS only.

These results demonstrated that one dose of Len17/H5 administered as either an LAIV or IIV provided substantial protection from infection, severe illness and death following challenge with the HP HK/483 virus. Additionally, both vaccines protected mice against replication of the antigenically variant HK/213 virus.

### EXAMPLE 12

### Immunogenicity and Cross-Reactive Antibody Cellular Responses Induced by H5 LAIV and IIV

Inactivated whole virus vaccines were prepared from Len17/H5 and HK/213 as previously described (Subbarao et al, 2003). A 2% suspension of alum was mixed with an equal volume of vaccine in PBS before immunization.

Eight-week-old female BALB/c mice (Jackson Laboratories, Bar Harbor, Mass., USA) were used in these experiments. Mice were immunized once by i.n. inoculation with Len17/H5 or Len17 (H2N2) as a control, or were immunized by i.m. inoculation with IIV prepared from Len17/H5 or HK/213 virus, administered with or without alum adjuvant.

Immune sera and nasal washes were collected as previously described (Katz et al, 1999). Sera were treated with receptor-destroying enzyme from Vibrio cholerae (Denka-Seiken, Tokyo, Japan) before testing for the presence of H5-specific antibodies (Kendal, 1982). Titres of neutralizing antibody were determined as previously described (Rowe et al, 1999).

An enzyme-linked immunosorbent assay (ELISA) was used for the detection of IgG, IgG1, IgG2a, and IgA antibodies in serum and/or nasal washes (Katz et al, 1999), except that 1 μg/ml of purified baculovirus-expressed recombinant H5 hemagglutinin (rHA; Protein Sciences Corporation, Meriden, Conn., USA) protein was used to coat the plates. The ELISA end-point titres were expressed as the highest dilution which yielded an optical density (OD) greater than twice the mean OD plus S.D. of similarly diluted negative control samples. Single spleen cell suspensions were prepared and stimulated with five hemagglutinating units (HAU) of formalin-inactivated whole H5 (HK/213) virus or 250 ng recombinant HA (HK/156) at a concentration of 5×10⁶ cells/ml (Lu et al, 2002). Culture supernatants were harvested after 5 days of culture. Interleukin (IL)-2, interferon (IFN)-γ, IL-4, and IL-10 were detected in culture supernatants by the Bio-Plex assay (BioRad Laboratories, Hercules, Calif.), used according to the manufacturer's instructions. The statistical significance of the data was determined using the Student's t-test.

Sera collected from mice 1 month after vaccination were tested for the presence of cross-reactive neutralizing antibodies against representative H5N1 viruses isolated from humans from 1997 to 2004. As shown in Table 10, neutralizing antibodies against the homologous virus were detected in sera from mice receiving Len17H5 LAIV inoculated by the i.n. route, but cross-reactive neutralizing antibodies against heterologous viruses HK/156, HK/213, and VN/1203 were not detected.

TABLE 10
Serum neutralizing antibody responses of mice immunized with H5
vaccines

| | | Neutralizing antibody titres^{b} | | | | |
|---|---|---|---|---|---|---|
| | | | Len17/ | | | VN/ |
| Vaccine group^{a} | Route | Len17 | H5 | HK/156 | HK/213 | 1203 |
| PBS | i.n. | <40^{c} | <40 | <40 | <40 | <40 |
| Len17 | i.n. | 80^{d} | <40 | <40 | <40 | <40 |
| Len17/H5 | i.n. | <40 | 80 | <40 | <40 | <40 |
| Len17/H5 | i.m. | <40 | 160 | 40 | <40 | <40 |
| Len17/H5 + alum | i.m. | <40 | 640 | 320 | 160 | <40 |
| HK/213 | i.m. | <40 | <40 | <40 | 320 | <40 |
| HK/213 + alum | i.m. | <40 | 40 | 80 | 5120 | 20 |

^{a}Mice were either infected i.n. with one dose of 300 MID₅₀ of LAIV or injected i.m. with one dose of 10 μg of IIV.
^{b}Sera were collected 1 month after vaccination and pooled from 10 mice per group to test neutralizing antibodies against H5 and H2 viruses.
^{c}Titres represent the reciprocal of the highest dilution of serum giving 50% neutralization of 100TCID₅₀ of virus. A titre of <40 represents the lower limit of detection.
^{d}Values in bold text represent titres to the homologous virus.

As expected, the Len17 ca H2N2 virus did not induce detectable cross-reactive neutralizing antibody against any H5 virus. Compared with H5 LAIV, Len17/H5 or HK/213 IIV induced at least two-fold higher serum neutralizing antibodies against homologous virus, but little if any cross-reactive antibody which could neutralize the heterologous human 1997 and 2004 H5N1 viruses. However, the addition of alum to either Len17/H5 or HK/213 IIV augmented the homologous antibody titres by 4-16-fold, and consequently also enhanced cross-reactive neutralizing antibody responses to heterologous H5N1 viruses.

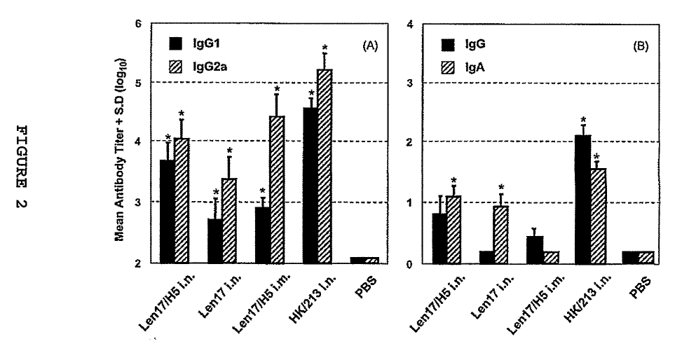

Levels of anti-H5 serum IgG and nasal wash IgG and IgA were determined by ELISA, and the results are illustrated in FIG. 2. Substantial levels of IgG1 and IgG2a which cross-reacted with VN/1203 HA were induced by both Len17/H5 LAIV and IIV, even though neutralizing antibodies against VN/1203 virus were not detected. Only LAIV vaccines administered by the i.n. route induced nasal IgA responses. Interestingly, both the Len17/H5 and Len17 (H2H2) LAIV induced nasal IgA responses which reacted to similar titres with H5 HA. In contrast, only Len17/H5 LAIV induced H5-specific nasal wash IgG at levels which were modestly higher than those elicited by Len17/H5 IIV.

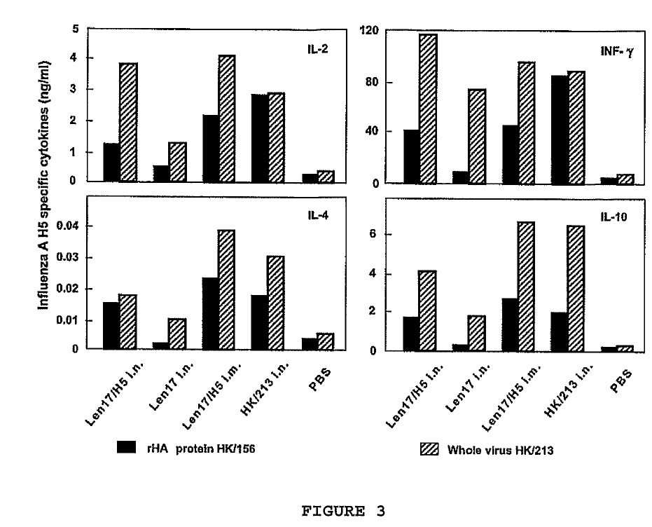

Cytokine production was evaluated in spleen cells isolated from mice immunized with LAIV or IIV which were restimulated in vitro with either H5 recombinant HA or inactivated whole H5N1 virus, and the results are shown in FIG. 3. Spleen cells from mice infected i.n. with wild-type HK/2I3 H5N1 virus, included as a positive control, produced either Th1-like (IFN-γ) or Th2-like (IL-4 and IL-10) cytokines. In all cases, stimulation of immune spleen cells with whole inactivated H5N1 virus induced more vigorous cytokine responses than stimulation with purified H5 recombinant HA. Compared with LAIV, IIV induced stronger IL-4 and IL-10 production when stimulated with inactivated whole H5N1 virus. Conversely, LAIV elicited higher levels of IFN-γ in spleen cells restimulated with whole virus, although differences were more modest. LAIV or IIV elicited similar levels of IL-2. However, mice administered Len17 (H2N2) LAIV produced primarily IFN-γ, and did so only when restimulated with whole H5N1 virus, suggesting that this subtype cross-reactive cellular response was directed against conserved epitopes on internal influenza A virus proteins. In contrast, the H5 LAIV or IIV responses were directed against epitopes present in H5 HA as well as against other viral proteins.

### EXAMPLE 13

### Cross-Protective Efficacy of H5 LAIV and IIV in Mice

We next evaluated the ability of Len17/H5 LAIV or IIV, and HK/213 IIV to protect mice from lethal challenges with 200 LD₅₀ VN/1203 virus, a HPAI H5N1 virus isolated from a fatal human case in early 2004 which is antigenically and genetically distinct from the vaccine strains.

To evaluate the degree of protection from lethal challenges, vaccinated mice were infected i.n. with 200 LD50 of VN/1203 virus. Mice were lightly anaesthetized with CO₂, and 50 μl of infectious virus diluted in PBS was inoculated i.n. Fifty percent mouse infectious dose (MID₅₀) and 50% lethal dose (LD₅₀) titres were determined as previously described (Lu et al, 1999). Five mice from each group were euthanized 6 days post infection (p.i.). Lung, nose and brain tissues were collected and titrated for virus infectivity as previously described (Lu et al, 1999). Virus titres were expressed as the mean log10 EID₅₀/ml±standard deviation (S.D.). The remaining mice in each group were observed daily for 14 days for weight loss and survival.

Groups of mice receiving one and/or two doses of H5 LAIV or IIV were challenged 3.5 months after the first vaccination or 2.5 months after second vaccination. Day 6 was chosen to evaluate the level of viral replication, because we have found that naive mice infected with VN/1203 have substantial titres of virus in the lung, nose and brain at this time point. As shown in Table 11, all control mice which received only PBS died 6-9 days after challenge with VN/1203 virus, having a mean maximum weight loss of 19% and high titres of virus in the lung, nose and brain on day 6 p.i.

TABLE 11
Cross-protective efficacy of H5 vaccines against 2004 H5N1 virus

| | | | | | | % Maximum | No. |
|---|---|---|---|---|---|---|---|
| Vaccine | Route/no. | Neut Ab | Mean virus titres^{c} | | | weight | died/no. |
| group^{a} | doses | (VN/1203)^{b} | Lung | Nose | Brain | loss^{d} | challenged^{d} |
| PBS | i.n./1 | <40 | 6.1 ± 0.7 | 4.7 ± 0.9 | 4.5 ± 0.1 | 19.4 | 5/5 |
| Len17 | i.n./1 | <40 | 5.6 ± 0.6^{e} | 1.0 ± 0.4 | 2.5 ± 1.7 | 21.6 | 1/5 |
| Len17/H5 | i.n./1 | <40 | 1.6 ± 1.2 | ≦0.8 | <0.8 | 7.3 | 0/5 |
| Len17/H5 | i.m./1 | <40 | 2.8 ± 1.8 | 1.0 ± 0.4 | 1.1 ± 0.7 | 11.2 | 0/7 |
| Len17/H5 | i.m./2 | <40 | ≦0.8 | ≦0.8 | ≦0.8 | 8.0 | 0/6 |
| Len17/H5 + alum | i.m./2 | <40 | ≦0.8 | ≦0.8 | ≦0.8 | 0.8 | 0/6 |
| HK/213 | i.m./2 | <40 | 1.5 ± 1.6 | ≦0.8 | ≦0.8 | 4.0 | 0/6 |
| HK/213 + alum | i.m./2 | 320 | ≦0.8 | ≦0.8 | ≦0.8 | 0.8 | 0/5 |

^{a}Mice were challenged i.n. with 200 LD₅₀ of a VN/1203 virus 3.5 months after the first vaccination or 2.5 months after the second vaccination.
^{b}Sera were collected before challenge and tested for neutralizing antibody (Neut Ab) against a VN/1203 virus.
^{c}Virus titres were determined 6 days p.i. and are expressed as the log₁₀ EID₅₀ /ml ± S.D. of five mice per group.
^{d}Mice were monitored daily for survival and weight loss for 14 days. Mice in PBS group died ≦7 days p.i.
^{e}All groups except that shown in bold text are p < 0.01 compared with PBS group.

Although 80% of mice immunized with Len17 H2N2 LAIV survived, the mice exhibited substantial weight loss and mean lung virus titres similar to those observed in unvaccinated control mice. On the other hand, viral titres in the upper respiratory tract and brain were significantly lower in mice which received the parent Len17 (H2N2) virus compared with those which received PBS (p<0.05). All mice immunized with one dose of Len17/H5 LAIV survived the lethal challenge, and exhibited only modest weight loss. Day 6 p.i. lung viral titres in mice immunized with Len17/H5 LAIV were more than 10,000-fold lower than those detected in mice immunized with Len17 LAIV or PBS; no virus was detected in the upper respiratory tract or brains of mice.

All mice immunized with one and two doses of Len17/H5 IIV or two doses of HK/213 IIV without alum adjuvant survived the lethal challenge with VN/1203 virus, but exhibited a modest weight loss. Low levels of virus were detected in the lung, nose and brain of mice administered one dose of Len17/H5 IIV and in the lungs of mice which received two doses of HK/213 IIV. Mice receiving two doses of either Len17/H5 or HK/213 IIV with alum adjuvant exhibited no disease signs, and no virus was isolated from any organs on 6 days p.i.

These results demonstrated that Len17/H5 administered as either a LAIV or IIV, and HK/2I3 administered as an IIV provided substantial cross-protection from infection, severe illness and death following challenge with a highly lethal heterologous human H5N1 2004 virus, although only the HK/2I3 IIV formulated with alum induced a detectable cross-reactive neutralizing antibody response against VN/1203 virus.

We have also demonstrated that the vaccine according to the invention indices cross-protective immunity in monkeys against infection with H5N1 influenza virus.

### Discussion

The optimal strategy for control of pandemic influenza is early intervention with a vaccine produced from the actual pandemic strain, or at least from a related strain which is a close antigenic match. In 2003 and 2004, inactivated vaccine strains were generated by reverse genetics, using the NA gene and HA gene modified to remove the multi-basic cleavage site motif from the wild-type HPAI H5N1 viruses and internal genes derived from A/PR/8/34, a high-growth donor strain for vaccine production in embryonated eggs. This approach requires the use of a high level of laboratory containment, safety testing of the recovered vaccine strain to ensure adequate attenuation for chickens and mammalian species, sophisticated patented reverse genetics technology, and a vaccine-qualified cell line. Furthermore, even in the best case scenario, it would take at least 6 months to produce an antigenically well-matched pandemic vaccine.

In this proof of concept study, we evaluated the immunogenicity and efficacy of a 7:1 reassortant H5 LAIV candidate generated from a non-pathogenic H5N2 strain, which is antigenically similar to the 1997 H5N1 viruses, and the Russian ca master donor strain Len17. Because the Len17/H5 vaccine candidate also possessed the high-growth properties in embryonated eggs which are desirable for the production of IIV, we also evaluated its utility as an IIV.

As an LAIV, a single dose of Len17/H5 induced superior H5 virus-specific IgA antibody responses in the respiratory tract, whereas a single dose of Len17/H5 IIV induced better cross-reactive serum neutralizing and IgG antibody responses to HK/156 virus HA. Surprisingly, a single dose of Len17/H5 administered either as an LAIV or IIV elicited protective immunity in mice against both related and antigenically variant H5N1 viruses.

LAIV against H5N1 viruses was first developed using reverse genetics technology to modify the HA from the HP H5N1 strains isolated from humans in Hong Kong in 1997. Two 6:2 reassortants were generated; these contained modified HA genes, and lacked the wild type neuraminidase (NA) genes from HK/156 and HK/483, the six internal gene segments from the attenuated ca A/Ann Arbor/6/60 donor strain, and the multibasic amino acid cleavage site associated with virulence in chickens. The resulting H5 LAIVs were not highly pathogenic for chickens, but gave variable immunity and protection in chickens following intravenous inoculation. However, the efficacy of these H5N1 LAIVs was not evaluated in mammals or humans (Li et al, 1999).

Another approach was used for the development of a surface antigen vaccine derived from a non-pathogenic H5N3 virus, antigenically related to the 1997 H5N1 strain. When evaluated in humans given two doses of the H5N3 IIV with or without MF-59 adjuvant, the non-adjuvanted IIV was poorly immunogenic, even after two doses of up to 30 μg of HA, whereas the adjuvanted H5N3 vaccine induced antibody titres which reached protective levels as measured by the single radial hemolysis assay (Nicholson et al, 2001).

Comparison of the amino acid sequences of the HA1 subunit demonstrated a 91-92% amino acid identity between the Len17/1-15 vaccine strain and the 1997 and 2003 H5N1 viruses used in the present study. Nevertheless, the Len17/H5 vaccines provided effective protection against H5N1 virus-induced death, severe disease and virus replication. As an LAIV, the Len17/H5 reassortant induced effective protection of mice against a lethal challenge with HK/483 virus, severe illness as measured by weight toss, and reduced lung viral titres by five logs at a time point when unvaccinated control mice succumbed to the lethal infection.

At this critical time point, no virus was detected in the upper respiratory tract or in systemic tissues of mice administered Len17/H5 LAIV. The lack of virus in the nose was associated with significant titres of H5-specific IgA in nasal washes. In fact, Len17/145 LAIV induced nasal and lung wash IgA titres which were comparable to those induced by infection with wild-type Pot/86 or HPAI HK/213 virus, whereas Len17/H5 IIV did not induce respiratory tract IgA responses. In contrast, serum neutralizing and IgG antibody against HK/156 were four-fold higher in mice which received Len17/H5 IIV, compared with those which received LAIV.

These results may explain the complete lack of detectable virus in lungs of mice which received IIV on day 6 p.i. Therefore, although Len17/H5 LAIV or IIV induced optimal responses in different antibody compartments, both vaccines provided substantial cross-protection following challenges with the 1997 and 2003 human H5N1 viruses. In Example 13, we have shown that the Len17/145 reassortant provides protection from lethal challenge with the recent highly pathogenic A/Vietnam/1203/2004 (H5N1) virus. While the Len17/H5 reassortant is immunogenic in ferrets, the extent to which it may replicate in and be immunogenic for humans has not yet been tested.

The NA genes of both parents used for preparation of Len17/145 were of the N2 subtype. It would require significant additional effort to select a 6:2 reassortant carrying the NA gene from the wild-type parent strain. Because time is limited if urgent preparation of a pandemic reassortant is needed, we studied the 7:1 reassortant vaccine which inherited the NA gene from the ca Len17 parent.

Our results have shown that an antigenically related NA was not essential for a protective effect against virulent H5N1 viruses in mice. However, other studies have demonstrated a role for NA-specific antibody in reducing the severity of disease in humans or in protecting mice from a lethal challenge with a mouse-adapted human influenza virus.

While a LAIV reassortant which possesses both HA and NA related to the circulating pandemic strain is desirable, it may not be appropriate for the N1 NA subtype, since some N1 gene products have been shown to enhance trypsin-independent cleavage of the HA molecule and thus could potentially lessen the attenuation of a live vaccine.

The use of LAIV in a pandemic situation has been considered previously. The generation of a cold-adapted influenza A H9N2 reassortant vaccine strain using classical reassortment techniques has been described, and clinical evaluation of such a candidate is ongoing. An important consideration in the use of a live-attenuated vaccine in the event of a pandemic is the potential for reassortment of the vaccine strain with the circulating strain bearing a novel HA. Therefore LAIV may be best used in a pandemic situation only when the population faces imminent widespread disease due to the novel wild-type pandemic strain.

Our results suggest a novel pandemic vaccine strategy which would allow for the stockpiling of an IIV which could be deployed immediately a pandemic strain had been identified. This would presumably be before widespread circulation of the virus, and certainly before a vaccine based on an exact antigenic match is available. If the pandemic strain became established in the population, the use of an LAIV generated from the same seed stock would extend the vaccine availability. An LAIV may have the added advantage of reducing viral shedding from the upper respiratory tract, which may be important for reducing transmission in a highly susceptible, immunologically naive population. Our results suggest a general strategy of using classical genetic reassortment between a high-growth ca H2N2 strain and antigenically related non-pathogenic avian viruses to prepare live attenuated and inactivated vaccines against multiple avian influenza A subtypes with pandemic potential. Similar strategies using non-pathogenic influenza viruses from other origins may be applied to influenza A subtypes with pandemic potential from the corresponding species.

We also evaluated an alternative strategy, which relied on the use of a nonpathogenic strain as the donor of the H5 HA gene and the generation of an attenuated reassortant virus with other genes from a ca master donor strain, using traditional reassortment methods. Our results demonstrate that one dose of Len17/H5 LAIV or two doses of IIV induced a high level of cross-protection from severe disease, death and viral replication, even though the vaccine and challenge strains shared an HA1 subunit amino acid identity of only 91%. Surprisingly, this cross-protective effect was observed in mice with low or undetectable levels of neutralizing antibodies against the challenge virus (Tables 10 and 11). However, nasal wash IgA and/or serum IgG which cross-reacted with VN/1203 HA were detected in mice which received LAIV and IIV, respectively (FIGS. 1 and 2). These results suggest a protective role for antibodies which are not detected by the serum neutralization assay.

The induction of cytokine-producing virus-specific T cells by either vaccine may also contribute to the broader cross-protective effect. Interestingly, the induction of cross-reactive nasal wash IgA antibody and T cell responses in mice which received Len17 (H2N2) LAIV was associated with protection from systemic spread of the virus and death, but not with reduction of lung viral titres and morbidity as measured by weight loss.

Nevertheless, the H5 subtype-specific response induced by any of the H5 vaccines was required for the reduction of virus load in the lungs and reduced morbidity.

Neither Len17/H5 LAIV nor IIV without adjuvant induced complete cross-protection from infection and illness, since a low level of viral replication and/or weight loss were detected in most of mice on day 6 after immunization. Only mice immunized with IIV formulated with adjuvant were found to have no detectable virus in any of the tissues tested on day 6 after immunization, and essentially no morbidity. Whether these results actually reflect complete cross-protection from infection in the respiratory tract remains to be established by determining the viral titres at an earlier time point after immunization.

The use of an adjuvant with IIV also augmented the H5 cross-reactive neutralizing antibody responses induced by both Len17/H5 and HK/213 IIV. Thus the use of an adjuvant in the formulation of pandemic vaccines may be particularly useful when an available IIV vaccine strain is not an optimal antigenic match with the circulating pandemic strain, or when reassortant virus stocks for vaccine production are limited.

We have demonstrated that an LAIV or adjuvanted IIV based on a heterologous H5 strain can significantly limit the disease severity and reduce mortality in mice following challenge with a contemporary HPAI H5N1 virus. These results suggest that heterotypic LAIV or adjuvanted IIV may provide a public health control measure to limit disease severity in the early stages of a pandemic prior to the availability of a strain-specific vaccine.

Subsequent to the filing of the priority application, 6:2, 7:1 and 5:3 influenza virus reassortants between A/Duck/Primorie/2621/2001 (H1N1) and A/Puerto Rico/8/34 have been reported (Rudneva et al, 2007). All three reassortants were pathogenic for mice. The 7:1 reassortant gave low yields in eggs, but a variant produced by serial passage in eggs produced yields comparable to those of the 6:2 reassortant. The 5:3 reassortant, produced by back-crossing the 6:2 reassortant with the parent strain, produced higher yields than either the 6:2 or the 7:1 reassortant. Only the 6:2 reassortant was tested for immunogenicity; this elicited an efficient protective response against a highly pathogenic H5N1 strain. The authors suggest that the back-crossing and selection of high-growth variants could be useful in production of highly pathogenic strains or reassortants of such strains, or to reassortants produced using reverse genetics. The use of low pathogenic strains is suggested in order to avoid the need to use reverse genetics. It is stated that the ability of the 6:2 reassortant to elicit protective immunity against an H5N1 strain “gives hope that this approach can be used for the preparation of “barricade” vaccines”.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

References cited herein are listed on the following pages, and are incorporated herein by this reference.

### REFERENCES

Alexandrova G J, Klimov A J Live Influenza Vaccine, Leningrad 1989 pp. 67-69
Alexandrova G. I. New in virus infections epidemiology and prophylactics. L., 1986 (p. 68-83).
Centers for Disease Control and Prevention. Outbreaks of avian influenza A (H5N1) in Asia and interim recommendations for evaluation and reporting of suspected cases-United States, 2004. MMWR 2004; 13(53):97-100.
Cox N J, Kendal A P. Genetic stability of A/Ann Arbor/6/60 cold-mutant (temperature-sensitive) live influenza virus genes: analysis by oligonucleotide mapping of recombinant vaccine strains before and after replication in volunteers, J Infect Dis 1984; 149: 194-200.
Ghendon Y Z, Polezhaev Fl, Lisovskaya K V, et al. Recombinant cold-adapted attenuated influenza A vaccines for use in children: molecular genetic analysis of the cold-adapted donor and recombinants. Infect Immun 1984; 44:730-3.
Ikizler M R and Wright P F Thermostabilization of egg grown influenza virus Vaccine 2002; 20:1393-9
Jones Y L, Swayne D E. Comparative pathobiology of low and high pathogenicity H7N3 Chilean avian influenza viruses in chickens. Avian Dis 2004; 48:1 19-28.
Katz J M, Lu X, Young S A, et al. Adjuvant activity of the heat-labile enterotoxin from enterotoxigenic Escherichia coli for oral administration of inactivated influenza virus vaccine, J Infect Dis 1997; 175:352-63.
Katz J M, Lim W, Bridges C E, et al. Antibody response in individuals infected with avian influenza A (H5N1) viruses and detection of anti-H5 antibody among household and social contacts. J Infect Dis 1999; 180: 1763-70.
Katz J M, Lu C W, Newman M J A non-ionic block copolymer adjuvant (CRL 1005) enhances the immunogenicity and protective efficacy of inactivated influenza vaccine in young and aged mice. Vaccine 2000; 18:2177-87
Kendal A P, Pereira M S. Skehel J Concepts and procedures for laboratory-based influenza surveillance. Atlanta: CDC; 1982. p. B 17-35.
Klimov A I, Cox N J J Virol Methods 1995 55: 445
Li S, Liu C. Klimov A, et al. Recombinant influenza A virus vaccine for the pathogenic human A/Hong Kong/97(H5N1) viruses. J Infect Dis 1999; 179:1132-8.
Lu X H, Tumpey T M, Morken T, et al. A mouse model for the evaluation of pathogenesis and immunity to influenza A (H5N1) viruses isolated from humans. J Virol 1999; 73:5903-11,
Lu X H, Clements J D, Katz J M. Mutant Escherichia coli heat-labile enterotoxin [LT (R192G)] enhances protective humoral and cellular immune responses to orally administered inactivated influenza vaccine. Vaccine 2002; 20:10J9-29,
Nicholson K G, Colegate A L, Podda A, et al. Safety and antigenicity of non-adjuvanted and MF59-adjuvanted influenza A/Duck/Singapore/97 (H5N3) vaccine: a randomised trial of two potential vaccines against H5N1 influenza. Lancet 2001; 357:1937-43.
Reed U, Muench H. A simple method of estimation 50% endpoints. Am J Hyg 1938; 27:493-7.
Rowe T, Abernathy R A, Hu-Primmer J, et al. Detection of antibody to avian influenza A (H5N1) virus in human serum by using a combination of serologic assays. J Clin Microbiol 1999; 37:937-43.
Rudneva I A, Timofeeva T A, Shilov A A, Kochergin-Nikitsky K S, Varich N L, Ilyushina N A, Gambaryan A S, Krylov P S, Kaverin N V. Effect of gene constellation and postreassortment amino acid change on the phenotypic features of H5 influenza virus reassortants. Arch Virol. 2007 Feb. 9; [Epub ahead of print]
Subbarao K, Chen I I, Swayne U, et al Evaluation of a genetically modified reassortant H5N1 influenza A virus vaccine candidate generated by plasmid-based reverse genetics. Virology 2003: 305:92-200.
Tumpey T M, Renshaw M, Clements J D, et al. Mucosal delivery of inactivated influenza vaccine induces B-cell-dependent heterosubtypic cross-protection against lethal influenza A H5N1 virus infection. J Virol 2001:75:5141-SO,
Van der Goot J A, G Koch, M C de Jong, and M van Boven Transmission dynamics of low- and high-pathogenicity A/Chicken/Pennsylvania/83 avian influenza viruses. Avian Dis, 2003; 47(3 Suppl): 939-41.
Van der Goot J A, De Jong M C M, Koch G, Van Boven M. Comparison of the transmission characteristics of low and high pathogenicity avian influenza A virus (H5N2). Epidemiol Infect 2003; 131:1003-13
Wareing M D, Marsh G A, Tannock G A.

Preparation and characterisation of attenuated cold-adapted influenza A reassortants derived from the A/Leningrad/′134/17/′57 donor strain. Vaccine 2002; 20:2082-2090.
Yannarell, D A, Goldberg, K M, Hjorth, R N Stabilizing cold-adapted influenza virus vaccine under various storage conditions. J Virol Methods 2002; 10:12-15

1. An influenza virus vaccine comprising
a) a reassortant influenza virus which has at least a hemagglutinin gene derived from a non-pathogenic avian influenza virus, and
b) other genes derived from a donor strain,
in which the non-pathogenic or low pathogenic influenza virus has the same hemagglutinin type as that of the highly pathogenic influenza virus.

2. A vaccine according to claim 1, in which the reassortant influenza virus is a 7:1 reassortant, in which only the hemagglutinin gene is derived from a non-pathogenic influenza virus.

3. A vaccine according to claim 1 or claim 2, in which the non-pathogenic or low pathogenic influenza virus is an avian virus.

4. A vaccine according to claim 3, in which the non-pathogenic or low pathogenic avian influenza virus is A/Duck/Potsdam/1042-6/86 (H5N2) A/Vietnam/1194/04(H5N1), A/Duck/Singapore/97 (H5N3), A/Duck/Hokkaido/67/96 (H5N4) or A/Mallard/Netherlands/12/00 (H7N3).

5. A vaccine according to any one of claims 1 to 4, in which the donor strain is a fully characterized vaccine strain.

6. A vaccine according to claim 5, in which the donor strain is a cold-adapted or temperature-sensitive strain.

7. A vaccine according to claim 5, in which the donor strain is cold-adapted and temperature-sensitive.

8. A vaccine according to claim 6 or claim 7, in which the donor strain has mutations in the PB2, PA, NA and M genes.

9. A vaccine according to claim 8, in which the donor strain is A/Leningrad/134/17/57 (H2N2), A/Leningrad/134/47/57 (H2N2), A/Leningrad/134/17/K7/57 (H2N2), A/Moscow/21/65 (H2N2), A/Moscow/21/17/65 (H2N2), A/Ann Arbor/6/60 (H2N2), A/Puerto Rico/8/34 (H1N1) or A/Puerto Rico/8/59/1 (H1N1).

10. A vaccine according to any one of claims 1 to 9, in which the reassortant influenza virus is obtained by classical reassortment.

11. A vaccine according to any one of claims 1 to 10, in which the reassortant influenza virus elicits a cross-protective immune response against a highly pathogenic influenza virus.

12. A vaccine according to any one of claims 1 to 11, which elicits IgA, IgG and T cell responses

13. A vaccine according to any one of claims 1 to 12, which is a live attenuated vaccine or an inactivated vaccine.

14. A vaccine according to claim 13, which is a live attenuated vaccine.

15. A vaccine according to claim 14, which is formulated for oral or intranasal administration.

16. A vaccine according to any one of claims 1 to 15, which also comprises an adjuvant.

17. A vaccine according to any one of claims 1 to 16, which also comprises one or more stabilizing agents which enable the vaccine to be stored at refrigerator temperature.

18. A vaccine according to any one of claims 1 to 17, which also comprises
(a) one or more additional influenza viruses, and/or
(b) a substantially pure influenza neuraminidase protein and/or influenza hemagglutinin protein.

19. A method of immunizing a subject against infection with a highly pathogenic influenza virus, comprising the step of administering a vaccine according to any one of claims 1 to 18 to the subject.

20. A method according to claim 19, in which the highly pathogenic influenza virus strain is an influenza virus strain of avian origin.

21. A method according to claim 19 or claim 20, in which the vaccine is a live attenuated vaccine.

22. A method according to claim 21, in which the vaccine is administered orally or intranasally.

23. A method according to any one of claims 19 to 22, which provides cross-protection and/or a cross-reactive immune response against a highly pathogenic influenza virus strain.

24. Use of an influenza virus as defined in any one of claims 1 to 18 in the manufacture of a vaccine for immunization of a subject against a highly pathogenic influenza virus strain.

25. Use according to claim 24, in which the vaccine is a live attenuated vaccine.

26. Use according to claim 24 or claim 25, in which the vaccine is formulated for oral or intranasal administration.

27. A reassortant influenza virus comprising a hemagglutinin gene derived from a non-pathogenic or low pathogenic influenza virus, and other genes derived from a donor strain, in which the non-pathogenic or low pathogenic influenza virus has the same hemagglutinin type as that of the highly pathogenic influenza virus.

28. A virus according to claim 27, which is a 7:1 reassortant, in which only the hemagglutinin gene is derived from a non-pathogenic influenza virus.

29. A virus according to claim 27 or claim 28, in which the non-pathogenic or low pathogenic influenza virus is an avian virus.

30. A virus according to claim 29, in which the non-pathogenic or low pathogenic avian influenza virus is A/Duck/Potsdam/1042-6/86 (H5N2) A/Vietnam/1194/04(H5N1), A/Duck/Singapore/97 (H5N3), A/Duck/Hokkaido/67/96 (H5N4) or A/Mallard/Netherlands/12/00 (H7N3).

31. A virus according to any one of claims 27 to 30, in which the donor strain is a fully characterized vaccine strain.

32. A virus according to any one of claims 27 to 31, in which the donor strain is a cold-adapted or temperature-sensitive strain.

33. A virus according to any one of claims 27 to 31, in which the donor strain is both cold-adapted and temperature-sensitive.

34. A virus according to any one of claims 27 to 33, in which the donor strain has mutations in the PB2, PA, NA and M genes.

35. A virus according to claim 31, in which the donor strain is A/Leningrad/134/17/57 (H2N2), A/Leningrad/134/47/57 (H2N2), A/Leningrad/134/17/K7/57 (H2N2), A/Moscow/21/65 (H2N2), A/Moscow/21/17/65 (H2N2), A/Ann Arbor/6/60 (H2N2), A/Puerto Rico/8/34 (H1N1) or A/Puerto Rico/8/59/1 (H1N1).

36. A virus according to any one of claims 27 to 35, which is obtained by classical reassortment.

37. A virus according to any one of claims 26 to 35, in which the reassortant influenza virus elicits a cross-protective immune response against a highly pathogenic influenza virus.

38. A virus according to claim 37, in which the highly pathogenic influenza virus against which the vaccine provides cross-protection is of hemagglutinin type H1, H2, H3, H4, H5, H6, H7, H8, H9, H10, H11, H12, H13, H14, H15 or H16.

39. A virus according to claim 37 or claim 38, in which the highly pathogenic influenza virus against which the vaccine provides cross-protection is of type H5N1, H5N2, H5N8, H5N9, H7N3, H7N7 or H9N2.

40. A method of preparing a vaccine for immunization of a subject against a highly pathogenic influenza virus strain, comprising the step of mixing a reassortant influenza virus according to any one of claims 26 to 38 with a carrier, and optionally an adjuvant.

41. A method according to claim 40, in which the vaccine also comprises one or more stabilizing agents which enable the vaccine to be stored at refrigerator temperature.

42. A method according to claim 40 or claim 41, in which the vaccine also comprises
(a) one or more other influenza viruses, and/or
(b) a substantially pure influenza neuraminidase protein and/or influenza hemagglutinin protein.

43. A method according to any one of claims 40 to 42, in which the vaccine is a live attenuated vaccine.

44. A method according to claim 43, in which the vaccine is formulated for oral or intranasal administration.

45. A method of making a reassortant influenza virus, which comprises a
a) a hemagglutinin gene derived from a non-pathogenic avian influenza virus, and
b) other genes derived from a donor strain,
comprising the step of subjecting a non-pathogenic or low pathogenic influenza virus which has the same hemagglutinin type as that of the highly pathogenic influenza virus to reassortment with a donor strain which has a different hemagglutinin type from that of the highly pathogenic influenza virus.

46. A method according to claim 45, in which the reassortment is classical reassortment.
